(19) United States
(12) Patent Application Publication
(10) Pub. No.: US 2024/0156749 A1
(43) Pub. Date: May 16, 2024

(54) Pre And Post Natal Compositions Including Carotenoids

(71) Applicant: Maravilla, LLC, Birmingham, MI (US)

(72) Inventor: James M. STRINGHAM, Phoenix, AZ (US)

(21) Appl. No.: 18/388,554

(22) Filed: Nov. 10, 2023

Related U.S. Application Data
(60) Provisional application No. 63/425,534, filed on Nov. 15, 2022.

(51) Int. Cl. A61K 31/047; A23L 33/00; A61K 9/00; A61K 31/07; A61K 31/14; A61K 31/197; A61K 31/202; A61K 31/355; A61K 31/375; A61K 31/4188; A61K 31/4415; A61K 31/455; A61K 31/51; A61K 31/519; A61K 31/525; A61K 31/593; A61K 31/714; A61K 33/04; A61K 33/06; A61K 33/18; A61K 33/26; A61K 33/30; A61K 33/32; A61K 33/34; A61K 36/9068

(52) U.S. Cl. CPC A61K 31/047; A23L 33/40; A61K 9/0056; A61K 31/07; A61K 31/14; A61K 31/197; A61K 31/202; A61K 31/355; A61K 31/375; A61K 31/4188; A61K 31/4415; A61K 31/455; A61K 31/51; A61K 31/519; A61K 31/525; A61K 31/593; A61K 31/714; A61K 33/04; A61K 33/06; A61K 33/18; A61K 33/26; A61K 33/30; A61K 33/32; A61K 33/34; A61K 36/9068

(57) ABSTRACT

A dietary supplement includes a xanthophyll carotenoid including lutein, zeaxanthin, and meso-zeaxanthin. The dietary supplement may include, for example, greater than or equal to about 0.27 weight percent to less than or equal to about 0.47 weight percent of lutein, greater than or equal to about 0.066 weight percent to less than or equal to about 0.84 weight percent of zeaxanthin, and greater than or equal to about 0.27 weight percent to less than or equal to about 0.47 weight percent of meso-zeaxanthin.

### CROSS-REFERENCE TO RELATED APPLICATIONS

This application claims the benefit of U.S. Provisional Application No. 63/425,534 filed on Nov. 15, 2022. The entire disclosure of the above application is incorporated herein by reference.

### FIELD

The present disclosure relates to compositions for optimizing visual and cognitive development in human fetuses and breastfeeding infants and to methods of making and using the same.

### BACKGROUND

This section provides background information related to the present disclosure which is not necessarily prior art.

It was recently determined that lutein and zeaxanthin are dominant carotenoids found in placentas of human subjects. Interestingly, however, the levels of lutein and zeaxanthin were found not to be correlated with the mother's current diet, suggesting instead that these dietary carotenoids accumulate, instead, over a period of time in the mother's adipose tissue and are subsequently released to support pregnancy and later breastfeeding. It has also been shown that during pregnancy, the mother's blood plasma levels of the dietary carotenoids can be directly correlated with levels in the umbilical cord. For example, at about gestation week six (which is before the fetus retina begins to develop), lutein and zeaxanthin may be transferred via the umbilical cord from the mother to the fetus, accumulating in an ocular reservoir, which is called the vitreous humor. At about gestation week twenty, as the retina begins to develop, lutein and zeaxanthin, and other carotenoids, may be transferred from the vitreous humor into the forming retinal tissue, where the carotenoids serve as antioxidants during the volatile and highly metabolic environment of neurogenesis and synaptogenesis. Oxygen is a major fuel for this metabolism, and the potential for free-radical oxidation stress and damage is high. Because of the timing of passage from the vitreous humor to the retina and the antioxidant capabilities of lutein and zeaxanthin, and other carotenoids, it is understood that lutein and zeaxanthin play a critical, early role in the protection and development of the retina, as well as other neural tissues, including, the brain.

Macular pigment, the accumulation of lutein, zeaxanthin, and meso-zeaxanthin in the central retina, may potentially mitigate the development and severity of retinopathy of prematurity (ROP), which is an eye disease that can occur in babies born prematurely or who weigh under about 3 pounds or 1.36 kilograms at birth. Macular pigment confers antioxidative and anti-inflammatory protection that counteracts oxidative stress and inflammation, which are known factors in retinopathy of prematurity development. Augmenting macular pigment status, especially in premature infants with lower levels, could potentially mitigate retinopathy of prematurity risk by providing enhanced protection against light-induced damage and retinal stress. It has been demonstrated in a mouse model of retinopathy of prematurity that prenatal supplementation of lutein or zeaxanthin significantly inhibits oxygen-induced retinopathy. Unlike full-term babies, macular pigment is undetectable in premature babies due to foveal immaturity and oxidative stress. Trials of postnatal macular pigment supplementation to prevent retinopathy of prematurity, however, were inconclusive. It is hypothesized that prenatal (vs. postnatal) supplementation provides time for accumulation of these key nutrients, which aids in slowing or stopping the onset of retinopathy of prematurity.

Because much development (and potential for oxidative stress) continues after birth, lutein and zeaxanthin, and other carotenoids, also play a role in the protection during the infant and childhood stages of development of the retina. Accordingly, it is desirable to provide compositions for consumption that can increase a mother's store of lutein and zeaxanthin, and other carotenoids during pregnancy, and also during post-partum recovery to restore and maintain carotenoid levels within breastmilk so that the infant can benefit from these important nutrients.

### SUMMARY

This section provides a general summary of the disclosure and is not a comprehensive disclosure of its full scope or all of its features.

The present disclosure provides a dietary supplement for human consumption.

In at least one example embodiment, the dietary supplement includes a xanthophyll carotenoid including lutein, zeaxanthin, and meso-zeaxanthin. The dietary supplement may include, for example, greater than or equal to about 0.27 weight percent to less than or equal to about 0.47 weight percent of lutein, greater than or equal to about 0.066 weight percent to less than or equal to about 0.84 weight percent of zeaxanthin, and greater than or equal to about 0.27 weight percent to less than or equal to about 0.47 weight percent of meso-zeaxanthin.

In at least one example embodiment, the weight ratio of lutein:zeaxanthin:meso-zeaxanthin may be about 5:1:5.

In at least one example embodiment, the dietary supplement may include greater than or equal to about 19.8 milligrams to less than or equal to about 24.2 milligrams of the xanthophyll carotenoid.

In at least one example embodiment, the dietary supplement may include greater than or equal to about 2 milligrams to less than or equal to about 11 milligrams of lutein, greater than or equal to about 0.4 milligrams to less than or equal to about 2 milligrams of zeaxanthin, and greater than or equal to about 2 milligrams to less than or equal to about 10 milligrams of meso-zeaxanthin.

In at least one example embodiment, the dietary supplement may further include Vitamin A, Vitamin C, Vitamin D3, Vitamin E, thiamin, riboflavin, niacin, Vitamin B6, folic acid, Vitamin B12, biotin, panthothenic acid, calcium, iron, iodine, magnesium, zinc, copper, choline, omega-3, selenium, manganese, ginger, or any combination thereof.

In at least one example embodiment, the dietary supplement may include greater than or equal to about 0.702 milligrams to less than or equal to about 0.858 milligrams of Vitamin A, greater than or equal to about 54 milligrams to less than or equal to about 66 milligrams of Vitamin C, greater than or equal to about 0.03375 milligrams to less than or equal to about 0.04125 milligrams of Vitamin D3, greater than or equal to about 18 milligrams to less than or equal to about 22 milligrams of Vitamin E, greater than or equal to about 1.53 milligrams to less than or equal to about 1.87 milligrams of thiamin, greater than or equal to about 1.98 milligrams to less than or equal to about 2.02 milligrams of riboflavin, greater than or equal to about 18 milligrams to less than or equal to about 22 milligrams of niacin, greater than or equal to about 1.8 milligrams to less than or equal to about 2.2 milligrams of Vitamin B6, greater than or equal to about 0.72 milligrams to less than or equal to about 0.88 milligrams of folic acid, greater than or equal to about 0.0072 milligrams to less than or equal to about 0.0088 milligrams of Vitamin B12, greater than or equal to about 0.045 milligrams to less than or equal to about 0.055 milligrams of biotin, greater than or equal to about 9 milligrams to less than or equal to about 11 milligrams of panthothenic acid, greater than or equal to about 450 milligrams to less than or equal to about 550 milligrams of calcium, greater than or equal to about 16.2 milligrams to less than or equal to about 19.8 milligrams of iron, greater than or equal to about 135 milligrams to less than or equal to about 165 milligrams of iodine, greater than or equal to about 45 milligrams to less than or equal to about 55 milligrams of magnesium, greater than or equal to about 0.9 milligrams to less than or equal to about 1.1 milligrams of zinc, greater than or equal to about 0.9 milligrams to less than or equal to about 1.1 milligrams of copper, greater than or equal to about 405 milligrams to less than or equal to about 495 milligrams of choline, greater than or equal to about 900 milligrams to less than or equal to about 1,100 milligrams of omega-3, greater than or equal to about 0.054 milligrams to less than or equal to about 0.066 milligrams of selenium, greater than or equal to about 1.98 milligrams to less than or equal to about 2.02 milligrams of manganese, and greater than or equal to about 339 milligrams to less than or equal to about 411 milligrams of ginger.

In at least one example embodiment, the omega-3 may include docosahexaenoic acid, eicosapentaenoic acid, or a combination of docosahexaenoic acid and eicosapentaenoic acid.

In at least one example embodiment, the dietary supplement may include about 0.78 milligrams of Vitamin A, about 60 milligrams of Vitamin C, about 0.0375 milligrams of Vitamin D3, about 20 milligrams of Vitamin E, about 1.7 milligrams of thiamin, about 2 milligrams of riboflavin, about 20 milligrams of niacin, about 2 milligrams of Vitamin B6, about 0.80 milligrams of folic acid, about 0.008 milligrams of Vitamin B12, about 0.05 milligrams of biotin, about 10 milligrams of panthothenic acid, about 500 milligrams of calcium, about 18 milligrams of iron, about 150 milligrams of iodine, about 50 milligrams of magnesium, about 11 milligrams of zinc, about 1 milligram of copper, about 450 milligrams of choline, about 500 milligrams of docosahexaenoic acid, about 500 milligrams of eicosapentaenoic acid, about 0.06 milligrams of selenium, about 2 milligrams of manganese, and about 375 milligrams of ginger.

In at least one example embodiment, the dietary supplement may further include greater than or equal to about 100 milligrams to less than or equal to about 300 milligrams of a filler.

In at least one example embodiment, the filler may include sunflower oil, micro-crystalline cellulose, silica, or any combination thereof.

In at least one example embodiment, the dietary supplement may include a first consumable. The first consumable may include lutein, meso-zeaxanthin, zeaxanthin, or any combination thereof.

In at least one example embodiment, the first consumable may include greater than or equal to about 2 milligrams to less than or equal to about 11 milligrams of lutein, greater than or equal to about 2 milligrams to less than or equal to about 10 milligrams of meso-zeaxanthin, and greater than or equal to about 0.4 milligrams to less than or equal to about 2 milligrams of zeaxanthin.

In at least one example embodiment, the first consumable may further include omega-3, Vitamin E, or a combination of omega-3 and Vitamin E.

In at least one example embodiment, the first consumable may include greater than or equal to about 450 milligrams to less than or equal to about 550 milligrams of omega-3 and greater than or equal to about 9 milligrams to less than or equal to about 11 milligrams of Vitamin E.

In at least one example embodiment, the dietary supplement may include a second consumable. The second consumable may include omega-3, Vitamin E, or a combination of omega-3 and Vitamin E.

In at least one example embodiment, the second consumable may include greater than or equal to about 450 milligrams to less than or equal to about 550 milligrams of omega-3 and greater than or equal to about 9 milligrams to less than or equal to about 11 milligrams of Vitamin E.

In at least one example embodiment, the dietary supplement may include a third consumable. The third consumable may include Vitamin D3, manganese, calcium, or any combination thereof.

In at least one example embodiment, the third consumable may include greater than or equal to about 0.03375 milligrams to less than or equal to about 0.04125 milligrams of Vitamin D3, greater than or equal to about 1.8 milligrams to less than or equal to about 2.2 milligrams of manganese, and greater than or equal to about 450 milligrams to less than or equal to about 550 milligrams of calcium.

In at least one example embodiment, the dietary supplement may include a fourth consumable. The fourth consumable may include Vitamin B1, Vitamin B2, niacin, biotin, pantothenic acid, iodine, magnesium, selenium, ginger, or any combination thereof.

In at least one example embodiment, the fourth consumable may include greater than or equal to about 1.53 milligrams to less than or equal to about 1.87 milligrams of Vitamin B1, greater than or equal to about 1.8 milligrams to less than or equal to about 2.2 milligrams of Vitamin B2, greater than or equal to about 18 milligrams to less than or equal to about 22 milligrams of niacin, greater than or equal to about 0.045 milligrams to less than or equal to about 0.055 milligrams of biotin, greater than or equal to about 9 milligrams to less than or equal to about 11 milligrams of pantothenic acid, greater than or equal to about 135 milligrams to less than or equal to about 165 milligrams of iodine, greater than or equal to about 45 milligrams to less than or equal to about 55 milligrams of magnesium, greater than or equal to about 0.054 milligrams to less than or equal to about 0.066 milligrams of selenium, and greater than or equal to about 315 milligrams to less than or equal to about 385 milligrams of ginger.

In at least one example embodiment, the dietary supplement may include a fifth consumable. The fifth consumable may include iron, Vitamin A, Vitamin C, Vitamin B6, Vitamin B12, choline, zinc, folic acid, copper, or any combination thereof.

In at least one example embodiment, the dietary supplement may include greater than or equal to about 16.2 milligrams to less than or equal to about 19.8 milligrams of iron, greater than or equal to about 0.702 milligrams to less than or equal to about 0.858 milligrams of Vitamin A, greater than or equal to about 54 milligrams to less than or equal to about 66 milligrams of Vitamin C, greater than or equal to about 1.8 milligrams to less than or equal to about 2.2 milligrams of Vitamin B6, greater than or equal to about 0.0072 milligrams to less than or equal to about 0.0088 milligrams of Vitamin B12, greater than or equal to about 405 milligrams to less than or equal to about 495 milligrams of choline, greater than or equal to about 9.9 milligrams to less than or equal to about 12.1 milligrams of zinc, greater than or equal to about 0.72 milligrams to less than or equal to about 0.88 milligrams of folic acid, and greater than or equal to about 0.9 milligrams to less than or equal to about 1.1 milligrams of copper.

Further areas of applicability will become apparent from the description provided herein. The description and specific examples in this summary are intended for purposes of illustration only and are not intended to limit the scope of the present disclosure.

### DETAILED DESCRIPTION

Example embodiments are provided so that this disclosure will be thorough and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

The terminology used herein is for the purpose of describing particular example embodiments only and is not intended to be limiting. As used herein, the singular forms “a,” “an,” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on,” “engaged to,” “connected to,” or “coupled to” another element or layer, it may be directly on, engaged, connected, or coupled to the other element or layer, or intervening elements or layers may be present. In contrast, when an element is referred to as being “directly on,” “directly engaged to,” “directly connected to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer, or section. Terms such as “first,” “second,” and other numerical terms when used herein do not imply a sequence or order unless clearly indicated by the context. Thus, a first element, component, region, layer, or section discussed below could be termed a second element, component, region, layer, or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

Dietary compositions or supplements (commonly in the form of tablets, capsules, gels, liquids, and/or powders) are often consumed by human subjects to supplement and/or replace important vitamins and nutrients that are not otherwise sufficiently absorbed via consumed foods. For example, mothers in various stages (including, for example, those who are expecting, as well as those who are who are actively trying to conceive and/or post-partum or recovering from birth or loss) often consume dietary supplements to counter the high consumption of certain vitamins and other nutrients by the mother and/or fetus or infant prior to, during, or after pregnancy. The mother may also consume dietary supplements having certain vitamins and other nutrients that have demonstrated importance for the developing fetus or infant.

In various aspects, the present disclosure provides dietary compositions for optimizing visual and cognitive development in human fetuses and breastfeeding infants. Compositions in accordance with various aspects of the present disclosure may be provided in any formulation suitable for oral consumption by a human subject, including, for example, as a tablet, capsule, gel, liquid, powder, and combinations and the like. In instances, where the example composition is provided in a capsule, the capsule may be prepared using, for example, hydroxypropyl methylcellulose, which is a clear, tasteless, and insert substance extracted from wood pulp, and magnesium stearate may be used, for example, as a lubricant to ensure that pieces of the capsule slide together.

Xanthophyll Carotenoids

An example composition prepared in accordance with the present disclosure includes a xanthophyll carotenoid.

In at least one example embodiment, the composition includes xanthophyll carotenoid in an amount greater than or equal to about 0.7 weight percent (e.g., greater than or equal to about 0.75 weight percent, greater than or equal to about 0.8 weight percent, greater than or equal to about 0.85 weight percent, greater than or equal to about 0.9 weight percent, or greater than or equal to about 0.95 weight percent). In at least one example embodiment, the composition includes xanthophyll carotenoid in an amount less than or equal to about 1 weight percent (e.g., less than or equal to about 0.95 weight percent, less than or equal to about 0.9 weight percent, less than or equal to about 0.85 weight percent, less than or equal to about 0.8 weight percent, or less than or equal to about 0.75 weight percent). In at least one example embodiment, the composition includes greater than or equal to about 0.7 weight percent to less than or equal to about 1 weight percent of xanthophyll carotenoid. In at least one example embodiment, the composition includes about 0.81 weight percent of the xanthophyll carotenoid.

In at least one example embodiment, the composition includes xanthophyll carotenoid in an amount greater than or equal to about 19.8 milligrams (e.g., greater than or equal to about 20 milligrams, greater than or equal to about 20.5 milligrams, greater than or equal to about 21 milligrams, greater than or equal to about 21.5 milligrams, greater than or equal to about 22 milligrams, greater than or equal to about 22.5 milligrams, greater than or equal to about 23 milligrams, greater than or equal to about 23.5 milligrams, or greater than or equal to about 24 milligrams). In at least one example embodiment, the composition includes xanthophyll carotenoid in an amount less than or equal to about 24.2 milligrams (e.g., less than or equal to about 24 milligrams, less than or equal to about 23.5 milligrams, less than or equal to about 23 milligrams, less than or equal to about 22.5 milligrams, less than or equal to about 22 milligrams, less than or equal to about 21.5 milligrams, less than or equal to about 21 milligrams, less than or equal to about 20.5 milligrams, or less than or equal to about 20 milligrams). In at least one example embodiment, the composition includes greater than or equal to about 19.8 milligrams to less than or equal to about 24.2 milligrams of xanthophyll carotenoid. In at least one example embodiment, the composition includes about 22 milligrams of the xanthophyll carotenoid.

The xanthophyll carotenoid may include, for example, lutein (L), zeaxanthin (Z), and/or meso-zeaxanthin (MZ). In at least one example embodiment, the example composition includes only one of lutein, zeaxanthin, and meso-zeaxanthin. In other example embodiments, the example composition includes, for example, two of lutein, zeaxanthin, and meso-zeaxanthin. In still other example embodiments, the example composition includes each of lutein, zeaxanthin, and meso-zeaxanthin. In at least one example embodiment, lutein may account for greater than or equal to about 25 percent to less than or equal to about 75 percent of the xanthophyll carotenoid. In at least one example embodiment, the zeaxanthin may account for greater than or equal to about 5 percent to less than or equal to about 25% of the xanthophyll carotenoid. In at least one example embodiment, the meso-zeaxanthin may account for greater than or equal to about 25 percent to less than or equal to about 75 percent of the xanthophyll carotenoid. In at least one example embodiment, the composition has a weight ratio of lutein:zeaxanthin:meso-zeaxanthin that is about 5:1:5.

Lutein. In at least one example embodiment, the composition includes lutein in an amount greater than or equal to about 0.27 weight percent (e.g., greater than or equal to about 0.3 weight percent, greater than or equal to about 0.35 weight percent, greater than or equal to about 0.4 weight percent, or greater than or equal to about 0.45 weight percent). In at least one example embodiment, the composition includes lutein in an amount less than or equal to about 0.47 weight percent (e.g., less than or equal to about 0.45 weight percent, less than or equal to about 0.4 weight percent, less than or equal to about 0.35 weight percent, or less than or equal to about 0.3 weight percent). In at least one example embodiment, the composition includes greater than or equal to about 0.27 weight percent to less than or equal to about 0.47 weight percent of lutein. In at least one example embodiment, the composition includes about 0.37 weight percent of lutein.

In at least one example embodiment, the composition includes lutein in an amount greater than or equal to about 2 milligrams (e.g., greater than or equal to about 2.5 milligrams, greater than or equal to about 3 milligrams, greater than or equal to about 3.5 milligrams, greater than or equal to about 4 milligrams, greater than or equal to about 4.5 milligrams, greater than or equal to about 5 milligrams, greater than or equal to about 5.5 milligrams, greater than or equal to about 6 milligrams, greater than or equal to about 6.5 milligrams, greater than or equal to about 7 milligrams, greater than or equal to about 7.5 milligrams, greater than or equal to about 8 milligrams, greater than or equal to about 8.5 milligrams, greater than or equal to about 9 milligrams, greater than or equal to about 9.5 milligrams, greater than or equal to about 10 milligrams, or greater than or equal to about 10.5 milligrams). In at least one example embodiment, the composition includes lutein in an amount less than or equal to about 11 milligrams (e.g., less than or equal to about 10.5 milligrams, less than or equal to about 10 milligrams, less than or equal to about 9.5 milligrams, less than or equal to about 9 milligrams, less than or equal to about 8.5 milligrams, less than or equal to about 8 milligrams, less than or equal to about 7.5 milligrams, less than or equal to about 7 milligrams, less than or equal to about 6.5 milligrams, less than or equal to about 6 milligrams, less than or equal to about 5.5 milligrams, less than or equal to about 5 milligrams, less than or equal to about 4.5 milligrams, less than or equal to about 4 milligrams, less than or equal to about 3.5 milligrams, less than or equal to about 3 milligrams, or less than or equal to about 2.5 milligrams). In at least one example embodiment, the composition includes greater than or equal to about 2 milligrams to less than or equal to about 11 milligrams, optionally greater than or equal to about 9 milligrams to less than or equal to about 11 milligrams, and optionally about 10 milligrams, of lutein.

Zeaxanthin. In at least one example embodiment, the composition includes zeaxanthin in an amount greater than or equal to about 0.066 weight percent (e.g., greater than or equal to about 0.07 weight percent, greater than or equal to about 0.075 weight percent, or greater than or equal to about 0.08 weight percent). In at least one example embodiment, the composition includes zeaxanthin in an amount less than or equal to about 0.081 weight percent (e.g., less than or equal to about 0.08 weight percent, less than or equal to about 0.075 weight percent, or less than or equal to about 0.07 weight percent). In at least one example embodiment, the composition includes greater than or equal to about 0.066 weight percent to less than or equal to about 0.84 weight percent of zeaxanthin. In at least one example embodiment, the composition includes about 0.74 weight percent of zeaxanthin.

In at least one example embodiment, the composition includes zeaxanthin in an amount greater than or equal to about 0.4 milligrams (e.g., greater than or equal to about 0.5 milligrams, greater than or equal to about 0.6 milligrams, greater than or equal to about 0.7 milligrams, greater than or equal to about 0.8 milligrams, greater than or equal to about 0.9 milligrams, greater than or equal to about 1 milligrams, greater than or equal to about 1.1 milligrams, greater than or equal to about 1.2 milligrams, greater than or equal to about 1.3 milligrams, greater than or equal to about 1.4 milligrams, greater than or equal to about 1.5 milligrams, greater than or equal to about 1.6 milligrams, greater than or equal to about 1.7 milligrams, greater than or equal to about 1.8 milligrams, or greater than or equal to about 1.9 milligrams). In at least one example embodiment, the composition includes zeaxanthin in an amount less than or equal to about 2 milligrams (e.g., less than or equal to about 1.9 milligrams, less than or equal to about 1.8 milligrams, less than or equal to about 1.7 milligrams, less than or equal to about 1.6 milligrams, less than or equal to about 1.5 milligrams, less than or equal to about 1.4 milligrams, less than or equal to about 1.3 milligrams, less than or equal to about 1.2 milligrams, less than or equal to about 1.1 milligrams, less than or equal to about 1 milligrams, less than or equal to about 0.9 milligrams, less than or equal to about 0.8 milligrams, less than or equal to about 0.7 milligrams, less than or equal to about 0.6 milligrams, of less than or equal to about 0.5 milligrams). In at least one example embodiment, the composition includes greater than or equal to about 0.4 milligrams to less than or equal to about 2 milligrams of zeaxanthin. In at least one example embodiment, the composition includes about 2 milligrams of zeaxanthin.

Meso-Zeaxanthin. In at least one example embodiment, the composition includes meso-zeaxanthin in an amount greater than or equal to about 0.27 weight percent (e.g., greater than or equal to about 0.3 weight percent, greater than or equal to about 0.35 weight percent, greater than or equal to about 0.4 weight percent, or greater than or equal to about 0.45 weight percent). In at least one example embodiment, the composition includes meso-zeaxanthin in an amount less than or equal to about 0.47 weight percent (e.g., less than or equal to about 0.45 weight percent, less than or equal to about 0.4 weight percent, less than or equal to about 0.35 weight percent, or less than or equal to about 0.3 weight percent). In at least one example embodiment, the composition includes greater than or equal to about 0.27 weight percent to less than or equal to about 0.47 weight percent of meso-zeaxanthin. In at least one example embodiment, the composition includes about 0.37 weight percent of meso-zeaxanthin.

In at least one example embodiment, the composition includes meso-zeaxanthin in an amount greater than or equal to about 2 milligrams (e.g., greater than or equal to about 2.5 milligrams, greater than or equal to about 3 milligrams, greater than or equal to about 3.5 milligrams, greater than or equal to about 4 milligrams, greater than or equal to about 4.5 milligrams, greater than or equal to about 5 milligrams, greater than or equal to about 5.5 milligrams, greater than or equal to about 6 milligrams, greater than or equal to about 6.5 milligrams, greater than or equal to about 7 milligrams, greater than or equal to about 7.5 milligrams, greater than or equal to about 8 milligrams, greater than or equal to about 8.5 milligrams, greater than or equal to about 9 milligrams, or greater than or equal to about 9.5 milligrams). In at least one example embodiment, the composition includes meso-zeaxanthin in an amount less than or equal to about 10 milligrams (e.g., less than or equal to about 9.5 milligrams, less than or equal to about 9 milligrams, less than or equal to about 8.5 milligrams, less than or equal to about 8 milligrams, less than or equal to about 7.5 milligrams, less than or equal to about 7 milligrams, less than or equal to about 6.5 milligrams, less than or equal to about 6 milligrams, less than or equal to about 5.5 milligrams, less than or equal to about 5 milligrams, less than or equal to about 4.5 milligrams, less than or equal to about 4 milligrams, less than or equal to about 3.5 milligrams, less than or equal to about 3 milligrams, or less than or equal to about 2.5 milligrams). In at least one example embodiment, the composition includes greater than or equal to about 2 milligrams to less than or equal to about 10 milligrams of meso-zeaxanthin. In at least one example embodiment, the composition includes about 10 milligrams of meso-zeaxanthin.

Other Active Ingredients

In at least one example embodiment, the composition further include one or more active ingredients. For example, the example composition may include, in addition to the lutein and/or zeaxanthin and/or meso-zeaxanthin, Vitamin A, Vitamin C, Vitamin D3, Vitamin E, thiamin (also referred to as Vitamin B1), riboflavin (also referred to as Vitamin B2), niacin, Vitamin B6, folic acid (for example, as methylfolate), Vitamin B12, biotin, panthothenic acid, calcium, iron (for example, in fermented bisglycinate form), iodine, magnesium, zinc, copper, choline (for example, as choline bitartrate), omega-3, docosahexaenoic acid (DHA), eicosapentaenoic acid (EPA), selenium, manganese, ginger (e.g., organic ginger), or any combination thereof.

Vitamin A. In at least one example embodiment, the composition includes Vitamin A in an amount greater than or equal to about 0.02601 weight percent (e.g., greater than or equal to about 0.027 weight percent, greater than or equal to about 0.028 weight percent, greater than or equal to about 0.029 weight percent, greater than or equal to about 0.03 weight percent, or greater than or equal to about 0.031 weight percent). In at least one example embodiment, the composition includes Vitamin A in an amount less than or equal to about 0.03179 weight percent (e.g., less than or equal to about 0.031 weight percent, less than or equal to about 0.03 weight percent, less than or equal to about 0.028 weight percent, or less than or equal to about 0.027 weight percent). In at least one example embodiment, the composition includes greater than or equal to about 0.02601 weight percent to less than or equal to about 0.03179 weight percent of Vitamin A. In at least one example embodiment, the composition includes about 0.0289 weight percent of Vitamin A.

In at least one example embodiment, the composition includes Vitamin A in an amount greater than or equal to about 0.702 milligrams (e.g., greater than or equal to about 0.71 milligrams, greater than or equal to about 0.72 milligrams, greater than or equal to about 0.73 milligrams, greater than or equal to about 0.74 milligrams, greater than or equal to about 0.75 milligrams, greater than or equal to about 0.76 milligrams, greater than or equal to about 0.77 milligrams, greater than or equal to about 0.78 milligrams, greater than or equal to about 0.79 milligrams, greater than or equal to about 0.8 milligrams, greater than or equal to about 0.81 milligrams, greater than or equal to about 0.82 milligrams, greater than or equal to about 0.83 milligrams, greater than or equal to about 0.84 milligrams, or greater than or equal to about 0.85 milligrams). In at least one example embodiment, the composition includes Vitamin A in an amount less than or equal to about 0.858 milligrams (e.g., less than or equal to about 0.85 milligrams, less than or equal to about 0.84 milligrams, less than or equal to about 0.83 milligrams, less than or equal to about 0.82 milligrams, less than or equal to about 0.81 milligrams, less than or equal to about 0.8 milligrams, less than or equal to about 0.79 milligrams, less than or equal to about 0.78 milligrams, less than or equal to about 0.77 milligrams, less than or equal to about 0.76 milligrams, less than or equal to about 0.75 milligrams, less than or equal to about 0.74 milligrams, less than or equal to about 0.73 milligrams, less than or equal to about 0.72 milligrams, or less than or equal to about 0.71 milligrams). In at least one example embodiment, the composition includes greater than or equal to about 0.702 milligrams to less than or equal to about 0.858 milligrams of Vitamin A. In at least one example embodiment, the composition includes about 0.78 milligrams of Vitamin A.

Vitamin C. In at least one example embodiment, the composition includes Vitamin C in an amount greater than or equal to about 2.12 weight percent (e.g., greater than or equal to about 2.15 weight percent, greater than or equal to about 2.2 weight percent, greater than or equal to about 2.25 weight percent, or greater than or equal to about 2.3 weight percent). In at least one example embodiment, the composition includes Vitamin C in an amount less than or equal to about 2.32 weight percent (e.g., less than or equal to about 2.3 weight percent, less than or equal to about 2.25 weight percent, less than or equal to about 2.2 weight percent, or less than or equal to about 2.15 weight percent). In at least one example embodiment, the composition includes greater than or equal to about 2.12 weight percent to less than or equal to about 2.32 weight percent of Vitamin C. In at least one example embodiment, the composition includes 2.22 weight percent.

In at least one example embodiment, the composition includes Vitamin C in an amount greater than or equal to about 54 milligrams (e.g., greater than or equal to about 55 milligrams, greater than or equal to about 56 milligrams, greater than or equal to about 57 milligrams, greater than or equal to about 58 milligrams, greater than or equal to about 59 milligrams, greater than or equal to about 60 milligrams, greater than or equal to about 61 milligrams, greater than or equal to about 62 milligrams, greater than or equal to about 63 milligrams, greater than or equal to about 64 milligrams, or greater than or equal to about 65 milligrams). In at least one example embodiment, the composition includes Vitamin C in an amount less than or equal to about 66 milligrams (e.g., less than or equal to about 65 milligrams, less than or equal to about 64 milligrams, less than or equal to about 63 milligrams, less than or equal to about 62 milligrams, less than or equal to about 61 milligrams, less than or equal to about 60 milligrams, less than or equal to about 59 milligrams, less than or equal to about 58 milligrams, less than or equal to about 57 milligrams, less than or equal to about 56 milligrams, or less than or equal to about 55 milligrams). In at least one example embodiment, the composition includes greater than or equal to about 54 milligrams to less than or equal to about 66 milligrams of Vitamin C. In at least one example embodiment, the composition includes about 60 milligrams of Vitamin C.

Vitamin D3. In at least one example embodiment, the composition includes Vitamin D3 in an amount greater than or equal to about 0.00125 weight percent (e.g., greater than or equal to about 0.0013 weight percent, greater than or equal to about 0.00135 weight percent, greater than or equal to about 0.0014 weight percent, greater than or equal to about 0.00145 weight percent, or greater than or equal to about 0.0015 weight percent). In at least one example embodiment, the composition includes Vitamin D3 in an amount less than or equal to about 0.00153 weight percent (e.g., less than or equal to about 0.0015 weight percent, less than or equal to about 0.00145 weight percent, less than or equal to about 0.0014 weight percent, less than or equal to about 0.00135 weight percent, or less than or equal to about 0.0013 weight percent). In at least one example embodiment, the composition includes greater than or equal to about 0.00125 weight percent to less than or equal to about 0.00153 weight percent of Vitamin D3. In at least one example embodiment, the composition includes about 0.00139 weight percent of Vitamin D3.

In at least one example embodiment, the composition includes Vitamin D3 in an amount greater than or equal to about 0.03375 milligrams (e.g., greater than or equal to about 0.034 milligrams, greater than or equal to about 0.035 milligrams, greater than or equal to about 0.036 milligrams, greater than or equal to about 0.037 milligrams, greater than or equal to about 0.038 milligrams, greater than or equal to about 0.039 milligrams, greater than or equal to about 0.04 milligrams, or greater than or equal to about 0.041 milligrams). In at least one example embodiment, the composition includes Vitamin D3 in an amount less than or equal to about 0.04125 milligrams (e.g., less than or equal to about 0.041 milligrams, less than or equal to about 0.04 milligrams, less than or equal to about 0.039 milligrams, less than or equal to about 0.038 milligrams, less than or equal to about 0.037 milligrams, less than or equal to about 0.036 milligrams, less than or equal to about 0.035 milligrams, or less than or equal to about 0.034 milligrams). In at least one example embodiment, the composition includes greater than or equal to about 0.03375 milligrams to less than or equal to about 0.04125 milligrams of Vitamin D3. In at least one example embodiment, the composition includes about 0.0375 milligrams of Vitamin D3.

Vitamin E. In at least one example embodiment, the composition includes Vitamin E in an amount greater than or equal to about 0.66 weight percent (e.g., greater than or equal to about 0.7 weight percent, greater than or equal to about 0.75 weight percent, or greater than or equal to about 0.8 weight percent). In at least one example embodiment, the composition includes Vitamin E in an amount less than or equal to about 0.81 weight percent (e.g., less than or equal to about 0.8 weight percent, less than or equal to about 0.75 weight percent, or less than or equal to about 0.7 weight percent). In at least one example embodiment, the composition includes greater than or equal to about 0.66 weight percent to less than or equal to about 0.81 weight percent of Vitamin E. In at least one example embodiment, the composition includes about 0.74 weight percent of Vitamin E.

In at least one example embodiment, the composition includes Vitamin E in an amount greater than or equal to about 18 milligrams (e.g., greater than or equal to about 18.2 milligrams, greater than or equal to about 18.4 milligrams, greater than or equal to about 18.6 milligrams, greater than or equal to about 18.8 milligrams, greater than or equal to about 19 milligrams, greater than or equal to about 19.2 milligrams, greater than or equal to about 19.4 milligrams, greater than or equal to about 19.6 milligrams, greater than or equal to about 19.8 milligrams, greater than or equal to about 20 milligrams, greater than or equal to about 20.2 milligrams, greater than or equal to about 20.4 milligrams, greater than or equal to about 20.6 milligrams, greater than or equal to about 20.8 milligrams, greater than or equal to about 21 milligrams, greater than or equal to about 21.2 milligrams, greater than or equal to about 21.4 milligrams, greater than or equal to about 21.6 milligrams, or greater than or equal to about 21.8 milligrams). In at least one example embodiment, the composition includes Vitamin E in an amount less than or equal to about 22 milligrams (e.g., less than or equal to about 21.8 milligrams, less than or equal to about 21.6 milligrams, less than or equal to about 21.4 milligrams, less than or equal to about 21.2 milligrams, less than or equal to about 21 milligrams, less than or equal to about 20.8 milligrams, less than or equal to about 20.6 milligrams, less than or equal to about 20.4 milligrams, less than or equal to about 20.2 milligrams, less than or equal to about 20 milligrams, less than or equal to about 19.8 milligrams, less than or equal to about 19.6 milligrams, less than or equal to about 19.4 milligrams, less than or equal to about 19.2 milligrams, less than or equal to about 19 milligrams, less than or equal to about 18.8 milligrams, less than or equal to about 18.6 milligrams, less than or equal to about 18.4 milligrams, or less than or equal to about 18.2 milligrams). In at least one example embodiment, the composition includes greater than or equal to about 18 milligrams to less than or equal to about 22 milligrams of Vitamin E. In at least one example embodiment, the composition includes about 20 milligrams of Vitamin E.

Thiamin. In at least one example embodiment, the composition includes thiamin in an amount greater than or equal to about 0.056 weight percent (e.g., greater than or equal to about 0.057 weight percent, greater than or equal to about 0.058 weight percent, greater than or equal to about 0.059 weight percent, greater than or equal to about 0.06 weight percent, greater than or equal to about 0.061 weight percent, greater than or equal to about 0.062 weight percent, greater than or equal to about 0.063 weight percent, greater than or equal to about 0.064 weight percent, greater than or equal to about 0.065 weight percent, greater than or equal to about 0.066 weight percent, greater than or equal to about 0.067 weight percent, or greater than or equal to about 0.068 weight percent). In at least one example embodiment, the composition includes thiamin in an amount less than or equal to about 0.069 weight percent (e.g., less than or equal to about 0.068 weight percent, less than or equal to about 0.067 weight percent, less than or equal to about 0.066 weight percent, less than or equal to about 0.065 weight percent, less than or equal to about 0.064 weight percent, less than or equal to about 0.063 weight percent, less than or equal to about 0.062 weight percent, less than or equal to about 0.061 weight percent, less than or equal to about 0.06 weight percent, less than or equal to about 0.059 weight percent, less than or equal to about 0.058 weight percent, or less than or equal to about 0.057 weight percent. In at least one example embodiment, the composition includes greater than or equal to about 0.056 weight percent to less than or equal to about 0.069 weight percent of thiamin. In at least one example embodiment, the composition includes about 0.063 weight percent of thiamin.

In at least one example embodiment, the composition includes thiamin in an amount greater than or equal to about 1.53 milligrams (e.g., greater than or equal to about 1.55 milligrams, greater than or equal to about 1.6 milligrams, greater than or equal to about 1.65 milligrams, greater than or equal to about 1.7 milligrams, greater than or equal to about 1.75 milligrams, greater than or equal to about 1.8 milligrams, or greater than or equal to about 1.85 milligrams). In at least one example embodiment, the composition includes thiamin in an amount less than or equal to about 1.87 milligrams (e.g., less than or equal to about 1.85 milligrams, less than or equal to about 1.8 milligrams, less than or equal to about 1.75 milligrams, less than or equal to about 1.7 milligrams, less than or equal to about 1.65 milligrams, less than or equal to about 1.6 milligrams, or less than or equal to about 1.55 milligrams). In at least one example embodiment, the composition includes greater than or equal to about 1.53 milligrams to less than or equal to about 1.87 milligrams of thiamin. In at least one example embodiment, the composition includes about 1.7 milligrams of thiamin.

Riboflavin. In at least one example embodiment, the composition includes riboflavin in an amount greater than or equal to about 0.066 weight percent (e.g., greater than or equal to about 0.070 weight percent, greater than or equal to about 0.075 weight percent, or greater than or equal to about 0.080 weight percent). In at least one example embodiment, the composition includes riboflavin in an amount less than or equal to about 0.081 weight percent (e.g., less than or equal to about 0.08 weight percent, less than or equal to about 0.075 weight percent, or less than or equal to about 0.07 weight percent). In at least one example embodiment, the composition includes greater than or equal to about 0.064 weight percent to less than or equal to about 0.081 weight percent of riboflavin. In at least one example embodiment, the composition includes about 0.074 weight percent of riboflavin.

In at least one example embodiment, the composition includes riboflavin in an amount greater than or equal to about 1.8 milligrams (e.g., greater than or equal to about 1.9 milligrams, greater than or equal to about 2 milligrams, or greater than or equal to about 2.1 milligrams). In at least one example embodiment, the composition includes riboflavin in an amount less than or equal to about 2.2 milligrams (e.g., less than or equal to about 2.1 milligrams, less than or equal to about 2 milligrams, or less than or equal to about 1.9 milligrams). In at least one example embodiment, the composition includes greater than or equal to about 1.98 milligrams to less than or equal to about 2.02 milligrams of riboflavin. In at least one example embodiment, the composition includes about 2 milligrams of riboflavin.

Niacin. In at least one example embodiment, the composition includes niacin in an amount greater than or equal to about 0.66 weight percent (e.g., greater than or equal to about 0.7 weight percent, greater than or equal to about 0.75 weight percent, or greater than or equal to about 0.8 weight percent). In at least one example embodiment, the composition includes niacin in an amount less than or equal to about 0.81 weight percent (e.g., less than or equal to about 0.8 weight percent, less than or equal to about 0.75 weight percent, or less than or equal to about 0.7 weight percent). In at least one example embodiment, the composition includes greater than or equal to about 0.66 weight percent to less than or equal to about 0.81 weight percent of niacin. In at least one example embodiment, the composition includes about 0.74 weight percent of niacin.

In at least one example embodiment, the composition includes niacin in an amount greater than or equal to about 18 milligrams (e.g., greater than or equal to about 18.5 milligrams, greater than or equal to about 19 milligrams, greater than or equal to about 19.5 milligrams, greater than or equal to about 20 milligrams, greater than or equal to about 20.5 milligrams, greater than or equal to about 21 milligrams, or greater than or equal to about 21.5 milligrams). In at least one example embodiment, the composition includes niacin in an amount less than or equal to about 22 milligrams (e.g., less than or equal to about 21.5 milligrams, less than or equal to about 21 milligrams, less than or equal to about 20.5 milligrams, less than or equal to about 20 milligrams, less than or equal to about 19.5 milligrams, less than or equal to about 19 milligrams, or less than or equal to about 18.5 milligrams). In at least one example embodiment, greater than or equal to about 18 milligrams to less than or equal to about 22 milligrams of niacin. In at least one example embodiment, the composition includes about 20 milligrams of niacin.

Vitamin B6. In at least one example embodiment, the composition includes Vitamin B6 in an amount greater than or equal to about 0.066 weight percent (e.g., greater than or equal to about 0.070 weight percent, greater than or equal to about 0.075 weight percent, or greater than or equal to about 0.080 weight percent). In at least one example embodiment, the composition includes Vitamin B6 in an amount less than or equal to about 0.081 weight percent (e.g., less than or equal to about 0.08 weight percent, less than or equal to about 0.075 weight percent, or less than or equal to about 0.07 weight percent). In at least one example embodiment, the composition includes greater than or equal to about 0.064 weight percent to less than or equal to about 0.081 weight percent of Vitamin B6. In at least one example embodiment, the composition includes about 0.074 weight percent of Vitamin B6.

In at least one example embodiment, the composition includes Vitamin B6 in an amount greater than or equal to about 1.8 milligrams (e.g., greater than or equal to about 1.9 milligrams, greater than or equal to about 2 milligrams, or greater than or equal to about 2.1 milligrams). In at least one example embodiment, the composition includes Vitamin B6 in an amount less than or equal to about 2.2 milligrams (e.g., less than or equal to about 2.1 milligrams, less than or equal to about 2 milligrams, or less than or equal to about 1.9 milligrams). In at least one example embodiment, the composition includes greater than or equal to about 1.8 milligrams to less than or equal to about 2.2 milligrams of Vitamin B6. In at least one example embodiment, the composition includes about 2 milligrams of Vitamin B6.

Folic Acid. In at least one example embodiment, the composition includes folic acid in an amount greater than or equal to about 0.0259 weight percent (e.g., greater than or equal to about 0.026 weight percent, greater than or equal to about 0.027 weight percent, greater than or equal to about 0.028 weight percent, greater than or equal to about 0.029 weight percent, greater than or equal to about 0.03 weight percent, greater than or equal to about 0.031 weight percent, greater than or equal to about 0.031 weight percent, or greater than or equal to about 0.032 weight percent). In at least one example embodiment, the composition includes folic acid in an amount less than or equal to about 0.0325 weight percent (e.g., less than or equal to about 0.032 weight percent, less than or equal to about 0.031 weight percent, less than or equal to about 0.03 weight percent, less than or equal to about 0.029 weight percent, less than or equal to about 0.028 weight percent, less than or equal to about 0.027 weight percent, or less than or equal to about 0.026 weight percent). In at least one example embodiment, the composition includes greater than or equal to about 0.0259 weight percent to less than or equal to about 0.0325 weight percent of folic acid. In at least one example embodiment, the composition includes about 0.0296 weight percent of folic acid.

In at least one example embodiment, the composition includes folic acid in an amount greater than or equal to about 0.72 milligrams (e.g., greater than or equal to about 0.73 milligrams, greater than or equal to about 0.74 milligrams, greater than or equal to about 0.75 milligrams, greater than or equal to about 0.76 milligrams, greater than or equal to about 0.77 milligrams, greater than or equal to about 0.78 milligrams, greater than or equal to about 0.79 milligrams, greater than or equal to about 0.8 milligrams, greater than or equal to about 0.81 milligrams, greater than or equal to about 0.82 milligrams, greater than or equal to about 0.83 milligrams, greater than or equal to about 0.84 milligrams, greater than or equal to about 0.85 milligrams, greater than or equal to about 0.86 milligrams, or greater than or equal to about 0.87 milligrams). In at least one example embodiment, the composition includes folic acid in an amount less than or equal to about 0.88 milligrams (e.g., less than or equal to about 0.87 milligrams, less than or equal to about 0.86 milligrams, less than or equal to about 0.85 milligrams, less than or equal to about 0.84 milligrams, less than or equal to about 0.83 milligrams, less than or equal to about 0.82 milligrams, less than or equal to about 0.81 milligrams, less than or equal to about 0.8 milligrams, less than or equal to about 0.79 milligrams, less than or equal to about 0.78 milligrams, less than or equal to about 0.77 milligrams, less than or equal to about 0.76 milligrams, less than or equal to about 0.75 milligrams, less than or equal to about 0.74 milligrams, or less than or equal to about 0.73 milligrams). In at least one example embodiment, the composition includes greater than or equal to about 0.72 milligrams to less than or equal to about 0.88 milligrams of folic acid. In at least one example embodiment, the composition includes about 0.80 milligrams of folic acid.

Vitamin B12. In at least one example embodiment, the composition includes Vitamin B12 in an amount greater than or equal to about 0.000333 weight percent (e.g., greater than or equal to about 0.00034 weight percent, greater than or equal to about 0.00035 weight percent, greater than or equal to about 0.00036 weight percent, greater than or equal to about 0.00037 weight percent, greater than or equal to about 0.00038 weight percent, greater than or equal to about 0.00039 weight percent, or greater than or equal to about 0.0004 weight percent). In at least one example embodiment, the composition includes Vitamin B12 in an amount less than or equal to about 0.000407 weight percent (e.g., less than or equal to about 0.0004 weight percent, less than or equal to about 0.00039 weight percent, less than or equal to about 0.00038 weight percent, less than or equal to about 0.00037 weight percent, less than or equal to about 0.00036 weight percent, less than or equal to about 0.00035 weight percent, or less than or equal to about 0.00034 weight percent). In at least one example embodiment, the composition includes greater than or equal to about 0.000333 weight percent to less than or equal to about 0.000407 weight percent of Vitamin B12. In at least one example embodiment, the composition includes 0.00037 weight percent of Vitamin B12.

In at least one example embodiment, the composition includes Vitamin B12 in an amount greater than or equal to about 0.0072 milligrams (e.g., greater than or equal to about 0.0075 milligrams, greater than or equal to about 0.008 milligrams, or greater than or equal to about 0.0085 milligrams). In at least one example embodiment, the composition includes Vitamin B12 in an amount less than or equal to about 0.0088 milligrams (e.g., less than or equal to about 0.0085 milligrams, less than or equal to about 0.008 milligrams, or less than or equal to about 0.0075 milligrams). In at least one example embodiment, the composition includes greater than or equal to about 0.0072 milligrams to less than or equal to about 0.0088 milligrams of Vitamin B12. In at least one example embodiment, the composition includes about 0.008 milligrams of Vitamin B12.

Biotin. In at least one example embodiment, the composition includes biotin in an amount greater than or equal to about 0.00166 weight percent (e.g., greater than or equal to about 0.0017 weight percent, greater than or equal to about 0.00175 weight percent, greater than or equal to about 0.0018 weight percent, greater than or equal to about 0.00185 weight percent, greater than or equal to about 0.0019 weight percent, or greater than or equal to about 0.00195 weight percent). In at least one example embodiment, the composition includes biotin in an amount less than or equal to about 0.00198 weight percent (e.g., less than or equal to about 0.00195 weight percent, less than or equal to about 0.0019 weight percent, less than or equal to about 0.00185 weight percent, less than or equal to about 0.0018 weight percent, less than or equal to about 0.00175 weight percent, or less than or equal to about 0.0017 weight percent). In at least one example embodiment, the composition includes greater than or equal to about 0.00166 weight percent to less than or equal to about 0.00198 weight percent of biotin. In at least one example embodiment, the composition includes about 0.0018 weight percent of biotin.

In at least one example embodiment, the composition includes biotin in an amount greater than or equal to about 0.045 milligrams (e.g., greater than or equal to about 0.046 milligrams, greater than or equal to about 0.047 milligrams, greater than or equal to about 0.048 milligrams, greater than or equal to about 0.049 milligrams, greater than or equal to about 0.05 milligrams, greater than or equal to about 0.051 milligrams, greater than or equal to about 0.052 milligrams, greater than or equal to about 0.053 milligrams, or greater than or equal to about 0.054 milligrams). In at least one example embodiment, the composition includes biotin in an amount less than or equal to about 0.055 milligrams (e.g., less than or equal to about 0.054 milligrams, less than or equal to about 0.053 milligrams, less than or equal to about 0.052 milligrams, less than or equal to about 0.051 milligrams, less than or equal to about 0.05 milligrams, less than or equal to about 0.049 milligrams, less than or equal to about 0.048 milligrams, less than or equal to about 0.047 milligrams, or less than or equal to about 0.046 milligrams). In at least one example embodiment, the composition includes greater than or equal to about 0.045 milligrams to less than or equal to about 0.055 milligrams of biotin. In at least one example embodiment, the composition includes about 0.05 milligrams of biotin.

Panthothenic Acid. In at least one example embodiment, the composition includes panthothenic acid in an amount greater than or equal to about 0.27 weight percent (e.g., greater than or equal to about 0.3 weight percent, greater than or equal to about 0.35 weight percent, greater than or equal to about 0.4 weight percent, or greater than or equal to about 0.45 weight percent). In at least one example embodiment, the composition includes panthothenic acid in an amount less than or equal to about 0.47 weight percent (e.g., less than or equal to about 0.45 weight percent, less than or equal to about 0.4 weight percent, less than or equal to about 0.35 weight percent, or less than or equal to about 0.3 weight percent). In at least one example embodiment, the composition includes greater than or equal to about 0.27 weight percent to less than or equal to about 0.47 weight percent of panthothenic acid. In at least one example embodiment, the composition includes about 0.37 weight percent of panthothenic acid.

In at least one example embodiment, the composition includes panthothenic acid in an amount greater than or equal to about 9 milligrams (e.g., greater than or equal to about 9.5 milligrams, greater than or equal to about 10 milligrams, or greater than or equal to about 10.5 milligrams). In at least one example embodiment, the composition includes panthothenic acid in an amount less than or equal to about 11 milligrams (e.g., less than or equal to about 10.5 milligrams, less than or equal to about 10 milligrams, or less than or equal to about 9.5 milligrams). In at least one example embodiment, the composition includes greater than or equal to about 9 milligrams to less than or equal to about 11 milligrams of panthothenic acid. In at least one example embodiment, the composition includes about 10 milligrams of panthothenic acid.

Calcium. In at least one example embodiment, the composition includes calcium in an amount greater than or equal to about 16 weight percent (e.g., greater than or equal to about 17 weight percent, greater than or equal to about 18 weight percent, greater than or equal to about 19 weight percent, or greater than or equal to about 20 weight percent). In at least one example embodiment, the composition includes calcium in an amount less than or equal to about 21 weight percent (e.g., less than or equal to about 20 weight percent, less than or equal to about 19 weight percent, less than or equal to about 18 weight percent, or less than or equal to about 17 weight percent). In at least one example embodiment, the composition includes greater than or equal to about 16 weight percent to less than or equal to about 21 weight percent of calcium. In at least one example embodiment, the composition includes about 18.5 weight percent of calcium.

In at least one example embodiment, the composition includes an amount of calcium greater than or equal to about 450 milligrams (e.g., greater than or equal to about 460 milligrams, greater than or equal to about 470 milligrams, greater than or equal to about 480 milligrams, greater than or equal to about 490 milligrams, greater than or equal to about 500 milligrams, greater than or equal to about 510 milligrams, greater than or equal to about 520 milligrams, greater than or equal to about 530 milligrams, or greater than or equal to about 540 milligrams). In at least one example embodiment, the composition includes an amount of calcium less than or equal to about 550 milligrams (e.g., less than or equal to about 540 milligrams, less than or equal to about 530 milligrams, less than or equal to about 520 milligrams, less than or equal to about 510 milligrams, less than or equal to about 500 milligrams, less than or equal to about 490 milligrams, less than or equal to about 480 milligrams, less than or equal to about 470 milligrams, or less than or equal to about 460 milligrams). In at least one example embodiment, the composition includes greater than or equal to about 450 milligrams to less than or equal to about 550 milligrams of calcium. In at least one example embodiment, the composition includes about 500 milligrams of calcium.

Iron. In at least one example embodiment, the composition includes iron in an amount greater than or equal to about 0.59 weight percent (e.g., greater than or equal to about 0.6 weight percent, greater than or equal to about 0.62 weight percent, greater than or equal to about 0.64 weight percent, greater than or equal to about 0.66 weight percent, greater than or equal to about 0.68 weight percent, or greater than or equal to about 0.7 weight percent). In at least one example embodiment, the composition includes iron in an amount less than or equal to about 0.72 weight percent (e.g., less than or equal to about 0.7 weight percent, less than or equal to about 0.68 weight percent, less than or equal to about 0.66 weight percent, less than or equal to about 0.64 weight percent, less than or equal to about 0.62 weight percent, or less than or equal to about 0.6 weight percent). In at least one example embodiment, the composition includes greater than or equal to about 0.59 weight percent to less than or equal to about 0.72 weight percent of iron. In at least one example embodiment, the composition includes about 0.66 weight percent of iron.

In at least one example embodiment, the composition includes iron in an amount greater than or equal to about 16.2 milligrams (e.g., greater than or equal to about 16.5 milligrams, greater than or equal to about 17 milligrams, greater than or equal to about 17.5 milligrams, greater than or equal to about 18 milligrams, greater than or equal to about 18.5 milligrams, greater than or equal to about 19 milligrams, or greater than or equal to about 19.5 milligrams. In at least one example embodiment, the composition includes iron in an amount less than or equal to about 19.8 milligrams (e.g., less than or equal to about 19.5 milligrams, less than or equal to about 19 milligrams, less than or equal to about 18.5 milligrams, less than or equal to about 18 milligrams, less than or equal to about 17.5 milligrams, less than or equal to about 17 milligrams, or less than or equal to about 16.5 milligrams). In at least one example embodiment, the composition includes greater than or equal to about 16.2 milligrams to less than or equal to about 19.8 milligrams of iron. In at least one example embodiment, the composition includes about 18 milligrams of iron.

Iodine. In at least one example embodiment, the composition includes iodine in an amount greater than or equal to about 4.9 weight percent (e.g., greater than or equal to about 5 weight percent, greater than or equal to about 5.2 weight percent, greater than or equal to about 5.4 weight percent, greater than or equal to about 5.6 weight percent, greater than or equal to about 5.8 weight percent, or greater than or equal to about 6 weight percent). In at least one example embodiment, the composition includes iodine in an amount less than or equal to about 6.1 weight percent (e.g., less than or equal to about 6 weight percent, less than or equal to about 5.8 weight percent, less than or equal to about 5.6 weight percent, less than or equal to about 5.4 weight percent, less than or equal to about 5.2 weight percent, or less than or equal to about 5 weight percent). In at least one example embodiment, the composition includes greater than or equal to about 4.9 weight percent to less than or equal to about 6.1 weight percent of iodine. In at least one example embodiment, the composition includes about 5.5 weight percent of iodine.

In at least one example embodiment, the composition includes iodine in an amount greater than or equal to about 135 milligrams (e.g., greater than or equal to about 140 milligrams, greater than or equal to about 145 milligrams, greater than or equal to about 150 milligrams, greater than or equal to about 155 milligrams, or greater than or equal to about 160 milligrams). In at least one example embodiment, the composition includes iodine in an amount less than or equal to about 165 milligrams (e.g., less than or equal to about 160 milligrams, less than or equal to about 155 milligrams, less than or equal to about 150 milligrams, less than or equal to about 145 milligrams, or less than or equal to about 140 milligrams). In at least one example embodiment, the composition includes greater than or equal to about 135 milligrams to less than or equal to about 165 milligrams of iodine. In at least one example embodiment, the composition includes about 150 milligrams of iodine.

Magnesium. In at least one example embodiment, the composition includes magnesium in an amount greater than or equal to about 1.66 weight percent (e.g., greater than or equal to about 1.7 weight percent, greater than or equal to about 1.8 weight percent, greater than or equal to about 1.9 weight percent, or greater than or equal to about 2 weight percent). In at least one example embodiment, the composition includes magnesium in an amount less than or equal to about 2.04 weight percent (e.g., less than or equal to about 2 weight percent, less than or equal to about 1.9 weight percent, less than or equal to about 1.8 weight percent, or less than or equal to about 1.7 weight percent). In at least one example embodiment, the composition includes greater than or equal to about 1.66 weight percent to less than or equal to about 2.04 weight percent of magnesium. In at least one example embodiment, the composition includes about 1.85 weight percent of magnesium.

In at least one example embodiment, the composition includes magnesium in an amount greater than or equal to about 45 milligrams (e.g., greater than or equal to about 46 milligrams, greater than or equal to about 47 milligrams, greater than or equal to about 48 milligrams, greater than or equal to about 49 milligrams, greater than or equal to about 50 milligrams, greater than or equal to about 51 milligrams, greater than or equal to about 52 milligrams, greater than or equal to about 53 milligrams, or greater than or equal to about 54 milligrams). In at least one example embodiment, the composition includes magnesium in an amount less than or equal to about 55 milligrams (e.g., less than or equal to about 54 milligrams, less than or equal to about 53 milligrams, less than or equal to about 52 milligrams, less than or equal to about 51 milligrams, less than or equal to about 50 milligrams, less than or equal to about 49 milligrams, less than or equal to about 48 milligrams, less than or equal to about 47 milligrams, or less than or equal to about 46 milligrams). In at least one example embodiment, the composition includes greater than or equal to about 45 milligrams to less than or equal to about 55 milligrams of magnesium. In at least one example embodiment, the composition includes about 50 milligrams of magnesium.

Zinc. In at least one example embodiment, the composition includes zinc in an amount greater than or equal to about 0.36 weight percent (e.g., greater than or equal to about 0.37 weight percent, greater than or equal to about 0.38 weight percent, greater than or equal to about 0.39 weight percent, greater than or equal to about 0.4 weight percent, greater than or equal to about 0.41 weight percent, greater than or equal to about 0.42 weight percent, or greater than or equal to about 0.43 weight percent). In at least one example embodiment, the composition includes zinc in an amount less than or equal to about 0.44 weight percent (e.g., less than or equal to about 0.43 weight percent, less than or equal to about 0.42 weight percent, less than or equal to about 0.41 weight percent, less than or equal to about 0.4 weight percent, less than or equal to about 0.39 weight percent, less than or equal to about 0.38 weight percent, or less than or equal to about 0.37 weight percent). In at least one example embodiment, the composition includes greater than or equal to about 0.36 weight percent to less than or equal to about 0.44 weight percent of zinc. In at least one example embodiment, the composition includes about 0.4 weight percent of zinc.

In at least one example embodiment, the composition includes zinc in an amount greater than or equal to about 0.9 milligrams (e.g., greater than or equal to about 0.95 milligrams, greater than or equal to about 1 milligrams, or greater than or equal to about 1.05 milligrams). In at least one example embodiment, the composition includes zinc in an amount less than or equal to about 1.1 milligrams (e.g., less than or equal to about 1.05 milligrams, less than or equal to about 1 milligrams, or less than or equal to about 0.95 milligrams). In at least one example embodiment, the composition includes greater than or equal to about 0.9 milligrams to less than or equal to about 1.1 milligrams of zinc. In at least one example embodiment, the composition includes about 11 milligrams of zinc.

Copper. In at least one example embodiment, the composition includes copper in an amount greater than or equal to about 0.3 weight percent (e.g., greater than or equal to about 0.32 weight percent, greater than or equal to about 0.34 weight percent, greater than or equal to about 0.36 weight percent, or greater than or equal to about 0.38 weight percent). In at least one example embodiment, the composition includes copper in an amount less than or equal to about 0.4 weight percent (e.g., less than or equal to about 0.38 weight percent, less than or equal to about 0.36 weight percent, less than or equal to about 0.34 weight percent, or less than or equal to about 0.32 weight percent). In at least one example embodiment, the composition includes greater than or equal to about 0.3 weight percent to less than or equal to about 0.4 weight percent of copper. In at least one example embodiment, the composition includes about 0.37 weight percent of copper.

In at least one example embodiment, the composition includes copper in an amount greater than or equal to about 0.9 milligrams (e.g., greater than or equal to about 0.95 milligrams, greater than or equal to about 1 milligrams, or greater than or equal to about 1.05 milligrams). In at least one example embodiment, the composition includes copper in an amount less than or equal to about 1.1 milligrams (e.g., less than or equal to about 1.05 milligrams, less than or equal to about 1 milligrams, or less than or equal to about 0.95 milligrams). In at least one example embodiment, the composition includes greater than or equal to about 0.9 milligrams to less than or equal to about 1.1 milligrams of copper. In at least one example embodiment, the composition includes about 1 milligram of copper.

Choline. In at least one example embodiment, the composition includes choline in an amount greater than or equal to about 14.9 weight percent (e.g., greater than or equal to about 15 weight percent, greater than or equal to about 15.5 weight percent, greater than or equal to about 16 weight percent, greater than or equal to about 16.5 weight percent, greater than or equal to about 17 weight percent, greater than or equal to about 17.5 weight percent, or greater than or equal to about 18 weight percent). In at least one example embodiment, the composition includes choline in an amount less than or equal to about 18.3 weight percent (e.g., less than or equal to about 18 weight percent, less than or equal to about 17.5 weight percent, less than or equal to about 17 weight percent, less than or equal to about 16.5 weight percent, less than or equal to about 16 weight percent, less than or equal to about 15.5 weight percent, or less than or equal to about 15 weight percent). In at least one example embodiment, the composition includes greater than or equal to about 14.9 weight percent to less than or equal to about 18.3 weight percent of choline. In at least one example embodiment, the composition includes about 16.6 weight percent of choline.

In at least one example embodiment, the composition includes choline in an amount greater than or equal to about 405 milligrams (e.g., greater than or equal to about 410 milligrams, greater than or equal to about 420 milligrams, greater than or equal to about 430 milligrams, greater than or equal to about 440 milligrams, greater than or equal to about 450 milligrams, greater than or equal to about 460 milligrams, greater than or equal to about 470 milligrams, greater than or equal to about 480 milligrams, or greater than or equal to about 490 milligrams). In at least one example embodiment, the composition includes choline in an amount less than or equal to about 495 milligrams (e.g., less than or equal to about 490 milligrams, less than or equal to about 480 milligrams, less than or equal to about 470 milligrams, less than or equal to about 460 milligrams, less than or equal to about 450 milligrams, less than or equal to about 440 milligrams, less than or equal to about 430 milligrams, less than or equal to about 420 milligrams, or less than or equal to about 410 milligrams). In at least one example embodiment, the composition includes greater than or equal to about 405 milligrams to less than or equal to about 495 milligrams of choline. In at least one example embodiment, the composition includes about 450 milligrams of choline.

Omega-3. In at least one example embodiment, the composition includes omega-3 in an amount greater than or equal to about 33 weight percent (e.g., greater than or equal to about 34 weight percent, greater than or equal to about 35 weight percent, greater than or equal to about 36 weight percent, greater than or equal to about 37 weight percent, greater than or equal to about 38 weight percent, greater than or equal to about 39 weight percent, or greater than or equal to about 40 weight percent). In at least one example embodiment, the composition includes omega-3 in an amount less than or equal to about 41 weight percent (e.g., less than or equal to about 40 weight percent, less than or equal to about 39 weight percent, less than or equal to about 38 weight percent, less than or equal to about 37 weight percent, less than or equal to about 36 weight percent, less than or equal to about 35 weight percent, or less than or equal to about 34 weight percent). In at least one example embodiment, the composition includes greater than or equal to about 33 weight percent to less than or equal to about 41 weight percent of omega-3. In at least one example embodiment, the composition includes about 37 weight percent of omega-3.

In at least one example embodiment, the composition includes greater than or equal to about 900 milligrams (e.g., greater than or equal to about 910 milligrams, greater than or equal to about 920 milligrams, greater than or equal to about 930 milligrams, greater than or equal to about 940 milligrams, greater than or equal to about 950 milligrams, greater than or equal to about 960 milligrams, greater than or equal to about 970 milligrams, greater than or equal to about 980 milligrams, greater than or equal to about 990 milligrams, greater than or equal to about 1,000 milligrams, greater than or equal to about 1,010 milligrams, greater than or equal to about 1,020 milligrams, greater than or equal to about 1,030 milligrams, greater than or equal to about 1,040 milligrams, greater than or equal to about 1,050 milligrams, greater than or equal to about 1,060 milligrams, greater than or equal to about 1,070 milligrams, greater than or equal to about 1,080 milligrams, or greater than or equal to about 1,090 milligrams). In at least one example embodiment, the composition includes less than or equal to about 1,100 milligrams (e.g., less than or equal to about 1,090 milligrams, less than or equal to about 1,080 milligrams, less than or equal to about 1,070 milligrams, less than or equal to about 1,060 milligrams, less than or equal to about 1,050 milligrams, less than or equal to about 1,040 milligrams, less than or equal to about 1,030 milligrams, less than or equal to about 1,020 milligrams, less than or equal to about 1,010 milligrams, less than or equal to about 1,000 milligrams, less than or equal to about 990 milligrams, less than or equal to about 980 milligrams, less than or equal to about 970 milligrams, less than or equal to about 960 milligrams, less than or equal to about 950 milligrams, less than or equal to about 940 milligrams, less than or equal to about 930 milligrams, less than or equal to about 920 milligrams, or less than or equal to about 910 milligrams). In at least one example embodiment, the composition includes greater than or equal to about 900 milligrams to less than or equal to about 1,100 milligrams of omega-3. In at least one example embodiment, the composition includes about 1,000 milligrams of omega-3.

In at least one example embodiment, the omega-3 includes docosahexaenoic acid (DHA), eicosapentaenoic acid (EPA), or a combination of docosahexaenoic acid (DHA) and eicosapentaenoic acid (EPA).

Docosahexaenoic acid (DHA). In at least one example embodiment, the composition includes docosahexaenoic acid (DHA) in an amount greater than or equal to about 16.6 weight percent (e.g., greater than or equal to about 17 weight percent, greater than or equal to about 17.5 weight percent, greater than or equal to about 18 weight percent, greater than or equal to about 18.5 weight percent, greater than or equal to about 19 weight percent, greater than or equal to about 19.5 weight percent, or greater than or equal to about 20 weight percent). In at least one example embodiment, the composition includes docosahexaenoic acid (DHA) in an amount less than or equal to about 20.3 weight percent (e.g., less than or equal to about 20 weight percent, less than or equal to about 19.5 weight percent, less than or equal to about 19 weight percent, less than or equal to about 18.5 weight percent, less than or equal to about 18 weight percent, less than or equal to about 17.5 weight percent, or less than or equal to about 17 weight percent). In at least one example embodiment, the composition includes greater than or equal to about 16.6 weight percent to less than or equal to about 20.3 weight percent of docosahexaenoic acid (DHA). In at least one example embodiment, the composition includes about 18.5 weight percent of docosahexaenoic acid (DHA).

In at least one example embodiment, the composition includes docosahexaenoic acid (DHA) in an amount greater than or equal to about 450 milligrams (e.g., greater than or equal to about 460 milligrams, greater than or equal to about 470 milligrams, greater than or equal to about 480 milligrams, greater than or equal to about 490 milligrams, greater than or equal to about 500 milligrams, greater than or equal to about 510 milligrams, greater than or equal to about 520 milligrams, greater than or equal to about 530 milligrams, or greater than or equal to about 540 milligrams). In at least one example embodiment, the composition includes docosahexaenoic acid (DHA) in an amount less than or equal to about 550 milligrams (e.g., less than or equal to about 540 milligrams, less than or equal to about 530 milligrams, less than or equal to about 520 milligrams, less than or equal to about 510 milligrams, less than or equal to about 500 milligrams, less than or equal to about 490 milligrams, less than or equal to about 480 milligrams, less than or equal to about 470 milligrams, or less than or equal to about 460 milligrams). In at least one example embodiment, the composition includes greater than or equal to about 450 milligrams to less than or equal to about 550 milligrams of docosahexaenoic acid (DHA). In at least one example embodiment, the composition includes about 500 milligrams of docosahexaenoic acid (DHA).

Eicosapentaenoic Acid (EPA). In at least one example embodiment, the composition includes eicosapentaenoic acid (EPA) in an amount 16.6 weight percent (e.g., greater than or equal to about 17 weight percent, greater than or equal to about 17.5 weight percent, greater than or equal to about 18 weight percent, greater than or equal to about 18.5 weight percent, greater than or equal to about 19 weight percent, greater than or equal to about 19.5 weight percent, or greater than or equal to about 20 weight percent). In at least one example embodiment, the composition includes eicosapentaenoic acid (EPA) in an amount less than or equal to about 20.3 weight percent (e.g., less than or equal to about 20 weight percent, less than or equal to about 19.5 weight percent, less than or equal to about 19 weight percent, less than or equal to about 18.5 weight percent, less than or equal to about 18 weight percent, less than or equal to about 17.5 weight percent, or less than or equal to about 17 weight percent). In at least one example embodiment, the composition includes greater than or equal to about 16.6 weight percent to less than or equal to about 20.3 weight percent of eicosapentaenoic acid (EPA). In at least one example embodiment, the composition includes about 18.5 weight percent of eicosapentaenoic acid (EPA).

In at least one example embodiment, the composition includes eicosapentaenoic acid (EPA) in an amount greater than or equal to about 450 milligrams (e.g., greater than or equal to about 460 milligrams, greater than or equal to about 470 milligrams, greater than or equal to about 480 milligrams, greater than or equal to about 490 milligrams, greater than or equal to about 500 milligrams, greater than or equal to about 510 milligrams, greater than or equal to about 520 milligrams, greater than or equal to about 530 milligrams, or greater than or equal to about 540 milligrams). In at least one example embodiment, the composition includes eicosapentaenoic acid (EPA) in an amount less than or equal to about 550 milligrams (e.g., less than or equal to about 540 milligrams, less than or equal to about 530 milligrams, less than or equal to about 520 milligrams, less than or equal to about 510 milligrams, less than or equal to about 500 milligrams, less than or equal to about 490 milligrams, less than or equal to about 480 milligrams, less than or equal to about 470 milligrams, or less than or equal to about 460 milligrams). In at least one example embodiment, the composition includes greater than or equal to about 450 milligrams to less than or equal to about 550 milligrams of eicosapentaenoic acid (EPA). In at least one example embodiment, the composition includes about 500 milligrams of eicosapentaenoic acid (EPA).

Selenium. In at least one example embodiment, the composition includes selenium in an amount greater than or equal to about 0.0017 weight percent (e.g., greater than or equal to about 0.0018 weight percent, greater than or equal to about 0.0019 weight percent, greater than or equal to about 0.002 weight percent, greater than or equal to about 0.0021 weight percent, greater than or equal to about 0.0022 weight percent, or greater than or equal to about 0.0023 weight percent). In at least one example embodiment, the composition includes selenium in an amount less than or equal to about 0.0024 weight percent (e.g., less than or equal to about 0.0023 weight percent, less than or equal to about 0.0022 weight percent, less than or equal to about 0.0021 weight percent, less than or equal to about 0.002 weight percent, less than or equal to about 0.0019 weight percent, or less than or equal to about 0.0018 weight percent). In at least one example embodiment, the composition includes greater than or equal to about 0.0017 weight percent to less than or equal to about 0.0024 weight percent of selenium. In at least one example embodiment, the composition includes about 0.0022 weight percent of selenium.

In at least one example embodiment, the composition includes greater than or equal to about 0.054 milligrams (e.g., greater than or equal to about 0.055 milligrams, greater than or equal to about 0.056 milligrams, greater than or equal to about 0.057 milligrams, greater than or equal to about 0.058 milligrams, greater than or equal to about 0.059 milligrams, greater than or equal to about 0.06 milligrams, greater than or equal to about 0.061 milligrams, greater than or equal to about 0.062 milligrams, greater than or equal to about 0.063 milligrams, greater than or equal to about 0.064 milligrams, or greater than or equal to about 0.065 milligrams). In at least one example embodiment, the composition includes less than or equal to about 0.066 milligrams (e.g., less than or equal to about 0.065 milligrams, less than or equal to about 0.064 milligrams, less than or equal to about 0.063 milligrams, less than or equal to about 0.062 milligrams, less than or equal to about 0.061 milligrams, less than or equal to about 0.06 milligrams, less than or equal to about 0.059 milligrams, less than or equal to about 0.058 milligrams, less than or equal to about 0.057 milligrams, less than or equal to about 0.056 milligrams, or less than or equal to about 0.055 milligrams). In at least one example embodiment, the composition includes greater than or equal to about 0.054 milligrams to less than or equal to about 0.066 milligrams of selenium. In at least one example embodiment, the composition includes about 0.06 milligrams of selenium.

Manganese. In at least one example embodiment, the composition includes manganese in an amount greater than or equal to about 0.066 weight percent (e.g., greater than or equal to about 0.070 weight percent, greater than or equal to about 0.075 weight percent, or greater than or equal to about 0.080 weight percent). In at least one example embodiment, the composition includes manganese in an amount less than or equal to about 0.081 weight percent (e.g., less than or equal to about 0.08 weight percent, less than or equal to about 0.075 weight percent, or less than or equal to about 0.07 weight percent). In at least one example embodiment, the composition includes greater than or equal to about 0.064 weight percent to less than or equal to about 0.081 weight percent of manganese. In at least one example embodiment, the composition includes about 0.074 weight percent of manganese.

In at least one example embodiment, the composition includes manganese in an amount greater than or equal to about 1.8 milligrams (e.g., greater than or equal to about 1.9 milligrams, greater than or equal to about 2 milligrams, or greater than or equal to about 2.1 milligrams). In at least one example embodiment, the composition includes manganese in an amount less than or equal to about 2.2 milligrams (e.g., less than or equal to about 2.1 milligrams, less than or equal to about 2 milligrams, or less than or equal to about 1.9 milligrams). In at least one example embodiment, the composition includes greater than or equal to about 1.98 milligrams to less than or equal to about 2.02 milligrams of manganese. In at least one example embodiment, the composition includes about 2 milligrams of manganese.

Ginger. In at least one example embodiment, the composition includes ginger in an amount greater than or equal to about 12.5 weight percent (e.g., greater than or equal to about 13 weight percent, greater than or equal to about 13.5 weight percent, greater than or equal to about 14 weight percent, greater than or equal to about 14.5 weight percent, or greater than or equal to about 15 weight percent). In at least one example embodiment, the composition includes ginger in an amount less than or equal to about 15.3 weight percent (e.g., less than or equal to about 15 weight percent, less than or equal to about 14.5 weight percent, less than or equal to about 14 weight percent, less than or equal to about 13.5 weight percent, or less than or equal to about 13 weight percent). In at least one example embodiment, the composition includes greater than or equal to about 12.5 weight percent to less than or equal to about 15.3 weight percent of ginger. In at least one example embodiment, the composition includes about 13.9 weight percent of ginger.

In at least one example embodiment, the composition includes greater than or equal to about 339 milligrams (e.g., greater than or equal to about 340 milligrams, greater than or equal to about 350 milligrams, greater than or equal to about 360 milligrams, greater than or equal to about 370 milligrams, greater than or equal to about 380 milligrams, greater than or equal to about 390 milligrams, greater than or equal to about 400 milligrams, or greater than or equal to about 410 milligrams). In at least one example embodiment, the composition includes ginger in an amount less than or equal to about 411 milligrams (e.g., less than or equal to about 410 milligrams, less than or equal to about 400 milligrams, less than or equal to about 390 milligrams, less than or equal to about 380 milligrams, less than or equal to about 370 milligrams, less than or equal to about 360 milligrams, less than or equal to about 350 milligrams, or less than or equal to about 340 milligrams). In at least one example embodiment, the composition includes greater than or equal to about 339 milligrams to less than or equal to about 411 milligrams of ginger. In at least one example embodiment, the composition includes about 375 milligrams of ginger.

Filler

In at least one example embodiment, the composition may further include a filler material. The filler material may help to stabilize and protect, and thereby enhance the effects of, the xanthophyll carotenoid. In at least one example embodiment, the filler material may include, for example, sunflower oil, micro-crystalline cellulose, silica, or any combination thereof.

In at least one example embodiment, the composition includes filler material in an amount greater than or equal to about 5 weight percent (e.g., greater than or equal to about 6 weight percent, greater than or equal to about 7 weight percent, greater than or equal to about 8 weight percent, greater than or equal to about 9 weight percent, greater than or equal to about 10 weight percent, greater than or equal to about 11 weight percent, greater than or equal to about 12 weight percent, greater than or equal to about 13 weight percent, greater than or equal to about 14 weight percent, greater than or equal to about 15 weight percent, greater than or equal to about 16 weight percent, greater than or equal to about 17 weight percent, greater than or equal to about 18 weight percent, greater than or equal to about 19 weight percent, greater than or equal to about 20 weight percent, greater than or equal to about 21 weight percent, greater than or equal to about 22 weight percent, greater than or equal to about 23 weight percent, greater than or equal to about 24 weight percent, greater than or equal to about 25 weight percent, greater than or equal to about 26 weight percent, greater than or equal to about 27 weight percent, greater than or equal to about 28 weight percent, greater than or equal to about 29 weight percent, greater than or equal to about 30 weight percent, greater than or equal to about 31 weight percent, greater than or equal to about 32 weight percent, greater than or equal to about 33 weight percent, or greater than or equal to about 34 weight percent). In at least one example embodiment, the composition includes filler material in an amount less than or equal to about 35 weight percent (e.g., less than or equal to about 34 weight percent, less than or equal to about 33 weight percent, less than or equal to about 32 weight percent, less than or equal to about 31 weight percent, less than or equal to about 30 weight percent, less than or equal to about 29 weight percent, less than or equal to about 28 weight percent, less than or equal to about 27 weight percent, less than or equal to about 26 weight percent, less than or equal to about 25 weight percent, less than or equal to about 24 weight percent, less than or equal to about 23 weight percent, less than or equal to about 22 weight percent, less than or equal to about 21 weight percent, less than or equal to about 20 weight percent, less than or equal to about 19 weight percent, less than or equal to about 18 weight percent, less than or equal to about 17 weight percent, less than or equal to about 16 weight percent, less than or equal to about 15 weight percent, less than or equal to about 14 weight percent, less than or equal to about 13 weight percent, less than or equal to about 12 weight percent, less than or equal to about 11 weight percent, less than or equal to about 10 weight percent, less than or equal to about 9 weight percent, less than or equal to about 8 weight percent, less than or equal to about 7 weight percent, or less than or equal to about 6 weight percent). In at least one example embodiment, the composition includes greater than or equal to about 5 weight percent to less than or equal to about 35 weight percent of filler material.

In at least one example embodiment, the composition includes filler material in an amount greater than or equal to about 100 milligrams (e.g., greater than or equal to about 110 milligrams, greater than or equal to about 120 milligrams, greater than or equal to about 130 milligrams, greater than or equal to about 140 milligrams, greater than or equal to about 150 milligrams, greater than or equal to about 160 milligrams, greater than or equal to about 170 milligrams, greater than or equal to about 180 milligrams, greater than or equal to about 190 milligrams, greater than or equal to about 200 milligrams, greater than or equal to about 210 milligrams, greater than or equal to about 220 milligrams, greater than or equal to about 230 milligrams, greater than or equal to about 240 milligrams, greater than or equal to about 250 milligrams, greater than or equal to about 260 milligrams, greater than or equal to about 270 milligrams, greater than or equal to about 280 milligrams, or greater than or equal to about 290 milligrams). In at least one example embodiment, the composition includes filler material in an amount less than or equal to about 300 milligrams (e.g., less than or equal to about 290 milligrams, less than or equal to about 280 milligrams, less than or equal to about 270 milligrams, less than or equal to about 260 milligrams, less than or equal to about 250 milligrams, less than or equal to about 240 milligrams, less than or equal to about 230 milligrams, less than or equal to about 220 milligrams, less than or equal to about 210 milligrams, less than or equal to about 200 milligrams, less than or equal to about 190 milligrams, less than or equal to about 180 milligrams, less than or equal to about 170 milligrams, less than or equal to about 160 milligrams, less than or equal to about 150 milligrams, less than or equal to about 140 milligrams, less than or equal to about 130 milligrams, less than or equal to about 120 milligrams, or less than or equal to about 110 milligrams). In at least one example embodiment, the composition includes greater than or equal to about 100 milligrams to less than or equal to about 300 milligrams of filler material. In at least one example embodiment, the composition includes about 180 milligrams of filler material.

Consumables

In at least one example embodiment, the composition in accordance with various aspects of the present disclosure may be provided (e.g., administered) in one or more consumables (e.g., tablets or pills or capsules), where each of the one or more consumables includes nutrients having similarly bioactivity (e.g., lipid v. water solubility). In at least one example embodiment, the example composition may be provided, for example, via a first consumable specifically formulated to support vision development and maintenance and/or a second consumable specifically formulated to support brain development and maintenance and/or a third consumable specifically formulated to support bone development and maintenance and/or a fourth consumable specifically formulated for the mother and/or a fifth consumable specifically formulated for general health of the fetus or infant and also the mother. Although five consumables are detailed below, it should be appreciated that in various other embodiments, a single consumable or two or more consumables may be provided. It should also be appreciated that although some examples are detailed below, in various embodiments, the consumables may include various combinations of the xanthophyll carotenoid and/or the other active ingredients and/or the fillers as detailed above.

### Example First Consumable

In at least one example embodiment, a first consumable of the one or more consumables includes, for example, lutein, meso-zeaxanthin, zeaxanthin, omega-3, Vitamin E, or any combination thereof. In at least one example embodiment, the omega-3 may include docosahexaenoic acid (DHA), eicosapentaenoic acid (EPA), or a combination of docosahexaenoic acid (DHA) and eicosapentaenoic acid (EPA).

In at least one example embodiment, a total weight of the first consumable is greater than or equal to about 500 milligrams (e.g., greater than or equal to about 510 milligrams, greater than or equal to about 520 milligrams, greater than or equal to about 530 milligrams, greater than or equal to about 540 milligrams, greater than or equal to about 550 milligrams, greater than or equal to about 560 milligrams, greater than or equal to about 570 milligrams, greater than or equal to about 580 milligrams, greater than or equal to about 590 milligrams, greater than or equal to about 600 milligrams, or greater than or equal to about 610 milligrams). In at least one example embodiment, a total weight of the first consumable is less than or equal to about 612 milligrams (e.g., less than or equal to about 610 milligrams, less than or equal to about 600 milligrams, less than or equal to about 590 milligrams, less than or equal to about 580 milligrams, less than or equal to about 570 milligrams, less than or equal to about 560 milligrams, less than or equal to about 550 milligrams, less than or equal to about 540 milligrams, less than or equal to about 530 milligrams, less than or equal to about 520 milligrams, or less than or equal to about 510 milligrams). In at least one example embodiment, a total weight of the first consumable is greater than or equal to about 500 milligrams to less than or equal to about 612 milligrams. In at least one example embodiment, a total weight of the first consumable is about 556 milligrams.

Lutein. In at least one example embodiment, the first consumable includes lutein in an amount greater than or equal to about 2 milligrams (e.g., greater than or equal to about 2.5 milligrams, greater than or equal to about 3 milligrams, greater than or equal to about 3.5 milligrams, greater than or equal to about 4 milligrams, greater than or equal to about 4.5 milligrams, greater than or equal to about 5 milligrams, greater than or equal to about 5.5 milligrams, greater than or equal to about 6 milligrams, greater than or equal to about 6.5 milligrams, greater than or equal to about 7 milligrams, greater than or equal to about 7.5 milligrams, greater than or equal to about 8 milligrams, greater than or equal to about 8.5 milligrams, greater than or equal to about 9 milligrams, greater than or equal to about 9.5 milligrams, greater than or equal to about 10 milligrams, or greater than or equal to about 10.5 milligrams). In at least one example embodiment, the first consumable includes lutein in an amount less than or equal to about 11 milligrams (e.g., less than or equal to about 10.5 milligrams, less than or equal to about 10 milligrams, less than or equal to about 9.5 milligrams, less than or equal to about 9 milligrams, less than or equal to about 8.5 milligrams, less than or equal to about 8 milligrams, less than or equal to about 7.5 milligrams, less than or equal to about 7 milligrams, less than or equal to about 6.5 milligrams, less than or equal to about 6 milligrams, less than or equal to about 5.5 milligrams, less than or equal to about 5 milligrams, less than or equal to about 4.5 milligrams, less than or equal to about 4 milligrams, less than or equal to about 3.5 milligrams, less than or equal to about 3 milligrams, or less than or equal to about 2.5 milligrams). In at least one example embodiment, the first consumable includes greater than or equal to about 2 milligrams to less than or equal to about 11 milligrams, optionally greater than or equal to about 9 milligrams to less than or equal to about 11 milligrams, and optionally about 10 milligrams, of lutein.

Meso-Zeaxanthin. In at least one example embodiment, the first consumable includes meso-zeaxanthin in an amount greater than or equal to about 2 milligrams (e.g., greater than or equal to about 2.5 milligrams, greater than or equal to about 3 milligrams, greater than or equal to about 3.5 milligrams, greater than or equal to about 4 milligrams, greater than or equal to about 4.5 milligrams, greater than or equal to about 5 milligrams, greater than or equal to about 5.5 milligrams, greater than or equal to about 6 milligrams, greater than or equal to about 6.5 milligrams, greater than or equal to about 7 milligrams, greater than or equal to about 7.5 milligrams, greater than or equal to about 8 milligrams, greater than or equal to about 8.5 milligrams, greater than or equal to about 9 milligrams, or greater than or equal to about 9.5 milligrams). In at least one example embodiment, the first consumable includes meso-zeaxanthin in an amount less than or equal to about 10 milligrams (e.g., less than or equal to about 9.5 milligrams, less than or equal to about 9 milligrams, less than or equal to about 8.5 milligrams, less than or equal to about 8 milligrams, less than or equal to about 7.5 milligrams, less than or equal to about 7 milligrams, less than or equal to about 6.5 milligrams, less than or equal to about 6 milligrams, less than or equal to about 5.5 milligrams, less than or equal to about 5 milligrams, less than or equal to about 4.5 milligrams, less than or equal to about 4 milligrams, less than or equal to about 3.5 milligrams, less than or equal to about 3 milligrams, or less than or equal to about 2.5 milligrams). In at least one example embodiment, the first consumable includes greater than or equal to about 2 milligrams to less than or equal to about 10 milligrams of meso-zeaxanthin. In at least one example embodiment, the first consumable includes about 10 milligrams of meso-zeaxanthin.

Zeaxanthin. In at least one example embodiment, the first consumable includes zeaxanthin in an amount greater than or equal to about 0.4 milligrams (e.g., greater than or equal to about 0.5 milligrams, greater than or equal to about 0.6 milligrams, greater than or equal to about 0.7 milligrams, greater than or equal to about 0.8 milligrams, greater than or equal to about 0.9 milligrams, greater than or equal to about 1 milligrams, greater than or equal to about 1.1 milligrams, greater than or equal to about 1.2 milligrams, greater than or equal to about 1.3 milligrams, greater than or equal to about 1.4 milligrams, greater than or equal to about 1.5 milligrams, greater than or equal to about 1.6 milligrams, greater than or equal to about 1.7 milligrams, greater than or equal to about 1.8 milligrams, or greater than or equal to about 1.9 milligrams). In at least one example embodiment, the first consumable includes zeaxanthin in an amount less than or equal to about 2 milligrams (e.g., less than or equal to about 1.9 milligrams, less than or equal to about 1.8 milligrams, less than or equal to about 1.7 milligrams, less than or equal to about 1.6 milligrams, less than or equal to about 1.5 milligrams, less than or equal to about 1.4 milligrams, less than or equal to about 1.3 milligrams, less than or equal to about 1.2 milligrams, less than or equal to about 1.1 milligrams, less than or equal to about 1 milligrams, less than or equal to about 0.9 milligrams, less than or equal to about 0.8 milligrams, less than or equal to about 0.7 milligrams, less than or equal to about 0.6 milligrams, of less than or equal to about 0.5 milligrams). In at least one example embodiment, the first consumable includes greater than or equal to about 0.4 milligrams to less than or equal to about 2 milligrams of zeaxanthin. In at least one example embodiment, the first consumable includes about 2 milligrams of zeaxanthin.

Omega-3. In at least one example embodiment, the first consumable includes omega-3 in an amount greater than or equal to about 450 milligrams (e.g., greater than or equal to about 460 milligrams, greater than or equal to about 470 milligrams, greater than or equal to about 480 milligrams, greater than or equal to about 490 milligrams, greater than or equal to about 500 milligrams, greater than or equal to about 510 milligrams, greater than or equal to about 520 milligrams, greater than or equal to about 530 milligrams, or greater than or equal to about 450 milligrams. In at least one example embodiment, the first consumable includes omega-3 in an amount less than or equal to about 550 milligrams (e.g., less than or equal to about 540 milligrams, less than or equal to about 530 milligrams, less than or equal to about 520 milligrams, less than or equal to about 510 milligrams, less than or equal to about 500 milligrams, less than or equal to about 490 milligrams, less than or equal to about 480 milligrams, less than or equal to about 470 milligrams, or less than or equal to about 460 milligrams). In at least one example embodiment, the first consumable includes greater than or equal to about 450 milligrams to less than or equal to about 550 milligrams of omega-3. In at least one example embodiment, the first consumable includes about 500 milligrams of omega-3.

Docosahexaenoic Acid (DHA). In at least one example embodiment, the first consumable includes docosahexaenoic acid (DHA) in an amount greater than or equal to about 225 milligrams (e.g., greater than or equal to about 230 milligrams, greater than or equal to about 235 milligrams, greater than or equal to about 240 milligrams, greater than or equal to about 245 milligrams, greater than or equal to about 250 milligrams, greater than or equal to about 255 milligrams, greater than or equal to about 260 milligrams, greater than or equal to about 265 milligrams, greater than or equal to about 270 milligrams). In at least one example embodiment, the first consumable includes docosahexaenoic acid (DHA) in an amount less than or equal to about 275 milligrams (e.g., less than or equal to about 270 milligrams, less than or equal to about 265 milligrams, less than or equal to about 260 milligrams, less than or equal to about 255 milligrams, less than or equal to about 250 milligrams, less than or equal to about 245 milligrams, less than or equal to about 240 milligrams, less than or equal to about 235 milligrams, or less than or equal to about 230 milligrams. In at least one example embodiment, the first consumable includes greater than or equal to about 225 milligrams to less than or equal to about 275 milligrams of docosahexaenoic acid (DHA). In at least one example embodiment, the first consumable includes about 250 milligrams of docosahexaenoic acid (DHA).

Eicosapentaenoic Acid (EPA). In at least one example embodiment, the first consumable includes eicosapentaenoic acid (EPA) in an amount greater than or equal to about 225 milligrams (e.g., greater than or equal to about 230 milligrams, greater than or equal to about 235 milligrams, greater than or equal to about 240 milligrams, greater than or equal to about 245 milligrams, greater than or equal to about 250 milligrams, greater than or equal to about 255 milligrams, greater than or equal to about 260 milligrams, greater than or equal to about 265 milligrams, greater than or equal to about 270 milligrams). In at least one example embodiment, the first consumable includes eicosapentaenoic acid (EPA) in an amount less than or equal to about 275 milligrams (e.g., less than or equal to about 270 milligrams, less than or equal to about 265 milligrams, less than or equal to about 260 milligrams, less than or equal to about 255 milligrams, less than or equal to about 250 milligrams, less than or equal to about 245 milligrams, less than or equal to about 240 milligrams, less than or equal to about 235 milligrams, or less than or equal to about 230 milligrams. In at least one example embodiment, the first consumable includes greater than or equal to about 225 milligrams to less than or equal to about 275 milligrams of eicosapentaenoic acid (EPA). In at least one example embodiment, the first consumable includes about 250 milligrams of eicosapentaenoic acid (EPA).

Vitamin E. In at least one example embodiment, the first consumable includes Vitamin E in an amount greater than or equal to about 9 milligrams (e.g., greater than or equal to about 9.5 milligrams, greater than or equal to about 10 milligrams, or greater than or equal to about 10.5 milligrams). In at least one example embodiment, the first consumable includes Vitamin E in an amount less than or equal to about 11 milligrams (e.g., less than or equal to about 10.5 milligrams, less than or equal to about 10 milligrams, or less than or equal to about 9.5 milligrams). In at least one example embodiment, the first consumable includes greater than or equal to about 9 milligrams to less than or equal to about 11 milligrams of Vitamin E. In at least one example embodiment, the first consumable includes about 10 milligrams of Vitamin E.

### Example Second Consumable

In at least one example embodiment, a second consumable of the one or more consumables includes, for example, omega-3 and Vitamin E. In at least one example embodiment, omega-3 includes docosahexaenoic acid (DHA), eicosapentaenoic acid (EPA), or a combination of docosahexaenoic acid (DHA) and eicosapentaenoic acid (EPA).

In at least one example embodiment, a total weight of the second consumable is greater than or equal to about 455 milligrams (e.g., greater than or equal to about 460 milligrams, greater than or equal to about 470 milligrams, greater than or equal to about 480 milligrams, greater than or equal to about 490 milligrams, greater than or equal to about 500 milligrams, greater than or equal to about 510 milligrams, greater than or equal to about 520 milligrams, greater than or equal to about 530 milligrams, greater than or equal to about 540 milligrams, greater than or equal to about 550 milligrams, or greater than or equal to about 560 milligrams). In at least one example embodiment, a total weight of the second consumable is less than or equal to about 565 milligrams (e.g., less than or equal to about 560 milligrams, less than or equal to about 550 milligrams, less than or equal to about 540 milligrams, less than or equal to about 530 milligrams, less than or equal to about 520 milligrams, less than or equal to about 510 milligrams, less than or equal to about 500 milligrams, less than or equal to about 490 milligrams, less than or equal to about 480 milligrams, less than or equal to about 470 milligrams, or less than or equal to about 460 milligrams). In at least one example embodiment, a total weight of the second consumable is greater than or equal to about 455 milligrams to less than or equal to about 565 milligrams. In at least one example embodiment, a total weight of the second consumable is about 510 milligrams.

Omega-3. In at least one example embodiment, the second consumable includes omega-3 in an amount greater than or equal to about 450 milligrams (e.g., greater than or equal to about 460 milligrams, greater than or equal to about 470 milligrams, greater than or equal to about 480 milligrams, greater than or equal to about 490 milligrams, greater than or equal to about 500 milligrams, greater than or equal to about 510 milligrams, greater than or equal to about 520 milligrams, greater than or equal to about 530 milligrams, or greater than or equal to about 450 milligrams. In at least one example embodiment, the second consumable includes omega-3 in an amount less than or equal to about 550 milligrams (e.g., less than or equal to about 540 milligrams, less than or equal to about 530 milligrams, less than or equal to about 520 milligrams, less than or equal to about 510 milligrams, less than or equal to about 500 milligrams, less than or equal to about 490 milligrams, less than or equal to about 480 milligrams, less than or equal to about 470 milligrams, or less than or equal to about 460 milligrams). In at least one example embodiment, the second consumable includes greater than or equal to about 450 milligrams to less than or equal to about 550 milligrams of omega-3. In at least one example embodiment, the second consumable includes about 500 milligrams of omega-3.

Docosahexaenoic Acid (DHA). In at least one example embodiment, the second consumable includes docosahexaenoic acid (DHA) in an amount greater than or equal to about 225 milligrams (e.g., greater than or equal to about 230 milligrams, greater than or equal to about 235 milligrams, greater than or equal to about 240 milligrams, greater than or equal to about 245 milligrams, greater than or equal to about 250 milligrams, greater than or equal to about 255 milligrams, greater than or equal to about 260 milligrams, greater than or equal to about 265 milligrams, greater than or equal to about 270 milligrams). In at least one example embodiment, the second consumable includes docosahexaenoic acid (DHA) in an amount less than or equal to about 275 milligrams (e.g., less than or equal to about 270 milligrams, less than or equal to about 265 milligrams, less than or equal to about 260 milligrams, less than or equal to about 255 milligrams, less than or equal to about 250 milligrams, less than or equal to about 245 milligrams, less than or equal to about 240 milligrams, less than or equal to about 235 milligrams, or less than or equal to about 230 milligrams. In at least one example embodiment, the second consumable includes greater than or equal to about 225 milligrams to less than or equal to about 275 milligrams of docosahexaenoic acid (DHA). In at least one example embodiment, the first consumable includes about 250 milligrams of docosahexaenoic acid (DHA).

Eicosapentaenoic Acid (EPA) In at least one example embodiment, the second consumable includes eicosapentaenoic acid (EPA) in an amount greater than or equal to about 225 milligrams (e.g., greater than or equal to about 230 milligrams, greater than or equal to about 235 milligrams, greater than or equal to about 240 milligrams, greater than or equal to about 245 milligrams, greater than or equal to about 250 milligrams, greater than or equal to about 255 milligrams, greater than or equal to about 260 milligrams, greater than or equal to about 265 milligrams, greater than or equal to about 270 milligrams). In at least one example embodiment, the second consumable includes eicosapentaenoic acid (EPA) in an amount less than or equal to about 275 milligrams (e.g., less than or equal to about 270 milligrams, less than or equal to about 265 milligrams, less than or equal to about 260 milligrams, less than or equal to about 255 milligrams, less than or equal to about 250 milligrams, less than or equal to about 245 milligrams, less than or equal to about 240 milligrams, less than or equal to about 235 milligrams, or less than or equal to about 230 milligrams. In at least one example embodiment, the second consumable includes greater than or equal to about 225 milligrams to less than or equal to about 275 milligrams of eicosapentaenoic acid (EPA). In at least one example embodiment, the second consumable includes about 250 milligrams of eicosapentaenoic acid (EPA).

Vitamin E. In at least one example embodiment, the first consumable includes Vitamin E in an amount greater than or equal to about 9 milligrams (e.g., greater than or equal to about 9.5 milligrams, greater than or equal to about 10 milligrams, or greater than or equal to about 10.5 milligrams). In at least one example embodiment, the second consumable includes Vitamin E in an amount less than or equal to about 11 milligrams (e.g., less than or equal to about 10.5 milligrams, less than or equal to about 10 milligrams, or less than or equal to about 9.5 milligrams). In at least one example embodiment, the second consumable includes greater than or equal to about 9 milligrams to less than or equal to about 11 milligrams of Vitamin E. In at least one example embodiment, the second consumable includes about 10 milligrams of Vitamin E.

### Example Third Consumable

In at least one example embodiment, a third consumable of the one or more consumables includes, for example Vitamin D3, manganese, calcium, or any combination thereof.

In at least one example embodiment, a total weight of the third consumable is greater than or equal to about 450 milligrams (e.g., greater than or equal to about 460 milligrams, greater than or equal to about 470 milligrams, greater than or equal to about 480 milligrams, greater than or equal to about 490 milligrams, greater than or equal to about 500 milligrams, greater than or equal to about 510 milligrams, greater than or equal to about 520 milligrams, greater than or equal to about 530 milligrams, or greater than or equal to about 540 milligrams). In at least one example embodiment, a total weight of the third consumable is less than or equal to about 550 milligrams (e.g., less than or equal to about 540 milligrams, less than or equal to about 530 milligrams, less than or equal to about 520 milligrams, less than or equal to about 510 milligrams, less than or equal to about 500 milligrams, less than or equal to about 490 milligrams, less than or equal to about 480 milligrams, less than or equal to about 470 milligrams, or less than or equal to about 460 milligrams). In at least one example embodiment, a total weight of the third consumable is greater than or equal to about 450 milligrams to less than or equal to about 550 milligrams. In at least one example embodiment, a total weight of the third consumable is about 502.04 milligrams.

Vitamin D3. In at least one example embodiment, the third consumable includes Vitamin D3 in an amount greater than or equal to about 0.03375 milligrams (e.g., greater than or equal to about 0.034 milligrams, greater than or equal to about 0.035 milligrams, greater than or equal to about 0.036 milligrams, greater than or equal to about 0.037 milligrams, greater than or equal to about 0.038 milligrams, greater than or equal to about 0.039 milligrams, or greater than or equal to about 0.04 milligrams). In at least one example embodiment, the third consumable includes Vitamin D3 in an amount less than or equal to about 0.04125 milligrams (e.g., less than or equal to about 0.04 milligrams, less than or equal to about 0.039 milligrams, less than or equal to about 0.038 milligrams, less than or equal to about 0.037 milligrams, less than or equal to about 0.036 milligrams, less than or equal to about 0.035 milligrams, or less than or equal to about 0.034 milligrams). In at least one example embodiment, the third consumable greater than or equal to about 0.03375 milligrams to less than or equal to about 0.04125 milligrams of Vitamin D3. In at least one example embodiment, the third consumable includes about 0.0375 milligrams of Vitamin D3.

Manganese. In at least one example embodiment, the third consumable includes manganese in an amount greater than or equal to about 1.8 milligrams (e.g., greater than or equal to about 1.9 milligrams, greater than or equal to about 2 milligrams, or greater than or equal to about 2.1 milligrams). In at least one example embodiment, the third consumable includes manganese in an amount less than or equal to about 2.2 milligrams (e.g., less than or equal to about 2.1 milligrams, less than or equal to about 2 milligrams, or less than or equal to about 1.9 milligrams. In at least one example embodiment, the third consumable includes greater than or equal to about 1.8 milligrams to less than or equal to about 2.2 milligrams of manganese. In at least one example embodiment, the third consumable includes about 2 milligrams of manganese.

Calcium. In at least one example embodiment, the third consumable includes calcium in an amount greater than or equal to about 450 milligrams (e.g., greater than or equal to about 460 milligrams, greater than or equal to about 470 milligrams, greater than or equal to about 480 milligrams, greater than or equal to about 490 milligrams, greater than or equal to about 500 milligrams, greater than or equal to about 510 milligrams, greater than or equal to about 520 milligrams, greater than or equal to about 530 milligrams, or greater than or equal to about 540 milligrams). In at least one example embodiment, the third consumable includes calcium in an amount less than or equal to about 550 milligrams (e.g., less than or equal to about 540 milligrams, less than or equal to about 530 milligrams, less than or equal to about 520 milligrams, less than or equal to about 510 milligrams, less than or equal to about 500 milligrams, less than or equal to about 490 milligrams, less than or equal to about 480 milligrams, less than or equal to about 470 milligrams, or less than or equal to about 460 milligrams). In at least one example embodiment, the third consumable includes greater than or equal to about 450 milligrams to less than or equal to about 550 milligrams of calcium. In at least one example embodiment, the third consumable includes about 500 milligrams of calcium.

### Example Fourth Consumable

In at least one example embodiment, a fourth consumable of the one or more consumables includes, for example, Vitamin B1, Vitamin B2, niacin, biotin, pantothenic acid, iodine, magnesium, selenium, ginger (e.g., organic ginger), or any combination thereof.

In at least one example embodiment, a total weight of the fourth consumable is greater than or equal to about 525 milligrams (e.g., greater than or equal to about 530 milligrams, greater than or equal to about 540 milligrams, greater than or equal to about 550 milligrams, greater than or equal to about 560 milligrams, greater than or equal to about 570 milligrams, greater than or equal to about 580 milligrams, greater than or equal to about 590 milligrams, greater than or equal to about 600 milligrams, greater than or equal to about 610 milligrams, greater than or equal to about 620 milligrams, or greater than or equal to about 630 milligrams). In at least one example embodiment, a total weight of the fourth consumable is less than or equal to about 640 milligrams (less than or equal to about 630 milligrams, less than or equal to about 620 milligrams, less than or equal to about 610 milligrams, less than or equal to about 600 milligrams, less than or equal to about 590 milligrams, less than or equal to about 580 milligrams, less than or equal to about 570 milligrams, less than or equal to about 560 milligrams, less than or equal to about 550 milligrams, less than or equal to about 540 milligrams, or less than or equal to about 530 milligrams). In at least one example embodiment, a total weight of the fourth consumable is greater than or equal to about 525 milligrams to less than or equal to about 640 milligrams. In at least one example embodiment, a total weight of the fourth consumable is about 583.81 milligrams.

Vitamin B1. In at least one example embodiment, the fourth consumable includes Vitamin B1 in an amount greater than or equal to about 1.53 milligrams (e.g., greater than or equal to about 1.55 milligrams, greater than or equal to about 1.6 milligrams, greater than or equal to about 1.65 milligrams, greater than or equal to about 1.7 milligrams, greater than or equal to about 1.75 milligrams, greater than or equal to about 1.8 milligrams, greater than or equal to about 1.85 milligrams). In at least one example embodiment, the fourth consumable includes Vitamin B1 in an amount less than or equal to about 1.87 milligrams (e.g., less than or equal to about 1.85 milligrams, less than or equal to about 1.8 milligrams, less than or equal to about 1.75 milligrams, less than or equal to about 1.7 milligrams, less than or equal to about 1.65 milligrams, less than or equal to about 1.6 milligrams, or less than or equal to about 1.55 milligrams). In at least one example embodiment, the fourth consumable includes greater than or equal to about 1.53 milligrams to less than or equal to about 1.87 milligrams of Vitamin B1. In at least one example embodiment, the fourth consumable includes about 1.7 milligrams of Vitamin B1.

Vitamin B2. In at least one example embodiment, the fourth consumable includes Vitamin B2 in an amount greater than or equal to about 1.8 milligrams (e.g., greater than or equal to about 1.85 milligrams, greater than or equal to about 1.9 milligrams, greater than or equal to about 1.95 milligrams, greater than or equal to about 2 milligrams, greater than or equal to about 2.05 milligrams, greater than or equal to about 2.1 milligrams, or greater than or equal to about 2.15 milligrams). In at least one example embodiment, the fourth consumable includes Vitamin B2 in an amount less than or equal to about 2.2 milligrams (e.g., less than or equal to about 2.15 milligrams, less than or equal to about 2.1 milligrams, less than or equal to about 2.05 milligrams, less than or equal to about 2 milligrams, less than or equal to about 1.95 milligrams, less than or equal to about 1.9 milligrams, or less than or equal to about 1.85 milligrams). In at least one example embodiment, the fourth consumable includes greater than or equal to about 1.8 milligrams to less than or equal to about 2.2 milligrams of Vitamin B2. In at least one example embodiment, the fourth consumable includes about 2 milligrams of Vitamin B2.

Niacin. In at least one example embodiment, the fourth consumable includes niacin in an amount greater than or equal to about 18 milligrams (e.g., greater than or equal to about 18.5 milligrams, greater than or equal to about 19 milligrams, greater than or equal to about 19.5 milligrams, greater than or equal to about 20 milligrams, greater than or equal to about 20.5 milligrams, greater than or equal to about 21 milligrams, or greater than or equal to about 21.5 milligrams). In at least one example embodiment, the fourth consumable includes niacin in an amount less than or equal to about 22 milligrams (e.g., less than or equal to about 21.5 milligrams, less than or equal to about 21 milligrams, less than or equal to about 20.5 milligrams, less than or equal to about 20 milligrams, less than or equal to about 19.5 milligrams, less than or equal to about 19 milligrams, or less than or equal to about 18.5 milligrams). In at least one example embodiment, the fourth consumable includes greater than or equal to about 18 milligrams to less than or equal to about 22 milligrams of niacin. In at least one example embodiment, the fourth consumable includes about 20 milligrams of niacin.

Biotin. In at least one example embodiment, the fourth consumable includes biotin in an amount greater than or equal to about 0.045 milligrams (e.g., greater than or equal to about 0.046 milligrams, greater than or equal to about 0.047 milligrams, greater than or equal to about 0.048 milligrams, greater than or equal to about 0.049 milligrams, greater than or equal to about 0.05 milligrams, greater than or equal to about 0.051 milligrams, greater than or equal to about 0.052 milligrams, greater than or equal to about 0.053 milligrams, or greater than or equal to about 0.054 milligrams). In at least one example embodiment, the fourth consumable includes biotin in an amount less than or equal to about 0.055 milligrams (e.g., less than or equal to about 0.054 milligrams, less than or equal to about 0.053 milligrams, less than or equal to about 0.052 milligrams, less than or equal to about 0.051 milligrams, less than or equal to about 0.05 milligrams, less than or equal to about 0.049 milligrams, less than or equal to about 0.048 milligrams, less than or equal to about 0.047 milligrams, or less than or equal to about 0.046 milligrams). In at least one example embodiment, the fourth consumable includes greater than or equal to about 0.045 milligrams to less than or equal to about 0.055 milligrams of biotin. In at least one example embodiment, the fourth consumable includes about 0.05 milligrams of biotin.

Pantothenic Acid. In at least one example embodiment, the fourth consumable includes pantothenic acid in an amount greater than or equal to about 9 milligrams (e.g., greater than or equal to about 9.5 milligrams, greater than or equal to about 10 milligrams, or greater than or equal to about 10.5 milligrams). In at least one example embodiment, the fourth consumable includes pantothenic acid in an amount less than or equal to about 11 milligrams (e.g., less than or equal to about 10.5 milligrams, less than or equal to about 10 milligrams, or less than or equal to about 9.5 milligrams). In at least one example embodiment, the fourth consumable includes greater than or equal to about 9 milligrams to less than or equal to about 11 milligrams of pantothenic acid. In at least one example embodiment, the fourth consumable includes about 10 milligrams of pantothenic acid.

Iodine. In at least one example embodiment, the fourth consumable includes iodine in an amount greater than or equal to about 135 milligrams (e.g., greater than or equal to about 140 milligrams, greater than or equal to about 145 milligrams, greater than or equal to about 150 milligrams, greater than or equal to about 155 milligrams, or greater than or equal to about 160 milligrams). In at least one example embodiment, the fourth consumable includes iodine in an amount less than or equal to about 165 milligrams (e.g., less than or equal to about 160 milligrams, less than or equal to about 155 milligrams, less than or equal to about 150 milligrams, less than or equal to about 145 milligrams, or less than or equal to about 140 milligrams). In at least one example embodiment, the fourth consumable includes greater than or equal to about 135 milligrams to less than or equal to about 165 milligrams of iodine. In at least one example embodiment, the fourth consumable includes about 150 milligrams of iodine.

Magnesium. In at least one example embodiment, the fourth consumable includes magnesium in an amount greater than or equal to about 45 milligrams (e.g., greater than or equal to about 46 milligrams, greater than or equal to about 47 milligrams, greater than or equal to about 48 milligrams, greater than or equal to about 49 milligrams, greater than or equal to about 50 milligrams, greater than or equal to about 51 milligrams, greater than or equal to about 52 milligrams, greater than or equal to about 53 milligrams, or greater than or equal to about 54 milligrams). In at least one example embodiment, the fourth consumable includes magnesium in an amount less than or equal to about 55 milligrams (e.g., less than or equal to about 54 milligrams, less than or equal to about 53 milligrams, less than or equal to about 52 milligrams, less than or equal to about 51 milligrams, less than or equal to about 50 milligrams, less than or equal to about 49 milligrams, less than or equal to about 48 milligrams, less than or equal to about 47 milligrams, or less than or equal to about 45 milligrams). In at least one example embodiment, the fourth consumable includes greater than or equal to about 45 milligrams to less than or equal to about 55 milligrams of magnesium. In at least one example embodiment, the fourth consumable includes about 50 milligrams of magnesium.

Selenium. In at least one example embodiment, the fourth consumable includes selenium in an amount greater than or equal to about 0.054 milligrams (e.g., greater than or equal to about 0.055 milligrams, greater than or equal to about 0.056 milligrams, greater than or equal to about 0.057 milligrams, greater than or equal to about 0.058 milligrams. greater than or equal to about 0.059 milligrams, greater than or equal to about 0.06 milligrams, greater than or equal to about 0.061 milligrams, greater than or equal to about 0.062 milligrams, greater than or equal to about 0.063 milligrams, greater than or equal to about 0.064 milligrams, or greater than or equal to about 0.065 milligrams). In at least one example embodiment, the fourth consumable includes selenium in an amount less than or equal to about 0.066 milligrams (e.g., less than or equal to about 0.065 milligrams, less than or equal to about 0.064 milligrams, less than or equal to about 0.063 milligrams, less than or equal to about 0.062 milligrams, less than or equal to about 0.061 milligrams, less than or equal to about 0.06 milligrams, less than or equal to about 0.059 milligrams, less than or equal to about 0.058 milligrams, less than or equal to about 0.057 milligrams, less than or equal to about 0.056 milligrams, or less than or equal to about 0.055 milligrams). In at least one example embodiment, the fourth consumable includes greater than or equal to about 0.054 milligrams to less than or equal to about 0.066 milligrams of selenium. In at least one example embodiment, the fourth consumable includes about 0.06 milligrams of selenium.

Ginger. In at least one example embodiment, the fourth consumable includes ginger in an amount greater than or equal to about 315 milligrams (e.g., greater than or equal to about 320 milligrams, greater than or equal to about 325 milligrams, greater than or equal to about 330 milligrams, greater than or equal to about 335 milligrams, greater than or equal to about 340 milligrams, greater than or equal to about 345 milligrams, greater than or equal to about 350 milligrams, greater than or equal to about 355 milligrams, greater than or equal to about 360 milligrams, greater than or equal to about 365 milligrams, greater than or equal to about 370 milligrams, greater than or equal to about 375 milligrams, or greater than or equal to about 380 milligrams). In at least one example embodiment, the fourth consumable includes ginger in an amount less than or equal to about 385 milligrams (e.g., less than or equal to about 380 milligrams, less than or equal to about 375 milligrams, less than or equal to about 370 milligrams, less than or equal to about 365 milligrams, less than or equal to about 360 milligrams, less than or equal to about 355 milligrams, less than or equal to about 350 milligrams, less than or equal to about 345 milligrams, less than or equal to about 340 milligrams, less than or equal to about 335 milligrams, less than or equal to about 330 milligrams, less than or equal to about 325 milligrams, or less than or equal to about 320 milligrams). In at least one example embodiment, the fourth consumable includes greater than or equal to about 315 milligrams to less than or equal to about 385 milligrams of ginger. In at least one example embodiment, the second consumable includes about 350 milligrams of ginger.

### Example Fifth Consumable

In at least one example embodiment, a fifth consumable of the one or more consumables includes, for example fermented iron bisglycinate, Vitamin A, Vitamin C, Vitamin B6, Vitamin B12, choline bitartrate, zinc, folic acid (as methlfolate), copper, or any combination thereof.

In at least one example embodiment, a total weight of the fifth consumable is greater than or equal to about 489 milligrams (e.g., greater than or equal to about 490 milligrams, greater than or equal to about 500 milligrams, greater than or equal to about 510 milligrams, greater than or equal to about 520 milligrams, greater than or equal to about 530 milligrams, greater than or equal to about 540 milligrams, greater than or equal to about 550 milligrams, greater than or equal to about 560 milligrams, greater than or equal to about 570 milligrams, greater than or equal to about 580 milligrams, or greater than or equal to about 590 milligrams). In at least one example embodiment, a total weight of the fifth consumable is less than or equal to about 599 milligrams (e.g., less than or equal to about 590 milligrams, less than or equal to about 580 milligrams, less than or equal to about 570 milligrams, less than or equal to about 560 milligrams, less than or equal to about 550 milligrams, less than or equal to about 540 milligrams, less than or equal to about 530 milligrams, less than or equal to about 520 milligrams, less than or equal to about 510 milligrams, less than or equal to about 500 milligrams, or less than or equal to about 490 milligrams). In at least one example embodiment, a total weight of the fifth consumable is greater than or equal to about 489 milligrams to less than or equal to about 599 milligrams. In at least one example embodiment, a total weight of the fifth consumable is about 543.59 milligrams.

Iron. In at least one example embodiment, the fifth consumable includes fermented iron bisglycinate in an amount greater than or equal to about 16.2 milligrams (e.g., greater than or equal to about 16.5 milligrams, greater than or equal to about 17 milligrams, greater than or equal to about 17.5 milligrams, greater than or equal to about 18 milligrams, greater than or equal to about 18.5 milligrams, greater than or equal to about 19 milligrams, or greater than or equal to about 19.5 milligrams). In at least one example embodiment, the fifth consumable includes fermented iron bisglycinate in an amount less than or equal to about 19.8 milligrams (e.g., less than or equal to about 19.5 milligrams, less than or equal to about 19 milligrams, less than or equal to about 18.5 milligrams, less than or equal to about 18 milligrams, less than or equal to about 17.5 milligrams, less than or equal to about 17 milligrams, or less than or equal to about 16.5 milligrams). In at least one example embodiment, the fifth consumable includes greater than or equal to about 16.2 milligrams to less than or equal to about 19.8 milligrams of fermented iron bisglycinate. In at least one example embodiment, the fifth consumable includes about 18 milligrams of fermented iron bisglycinate.

Vitamin A. In at least one example embodiment, the fifth consumable includes Vitamin A in an amount greater than or equal to about 0.702 milligrams (e.g., greater than or equal to about 0.71 milligrams, greater than or equal to about 0.72 milligrams, greater than or equal to about 0.73 milligrams, greater than or equal to about 0.74 milligrams, greater than or equal to about 0.75 milligrams, greater than or equal to about 0.76 milligrams, greater than or equal to about 0.77 milligrams, greater than or equal to about 0.78 milligrams, greater than or equal to about 0.79 milligrams, greater than or equal to about 0.8 milligrams, greater than or equal to about 0.81 milligrams, greater than or equal to about 0.82 milligrams, greater than or equal to about 0.83 milligrams, greater than or equal to about 0.84 milligrams, or greater than or equal to about 0.85 milligrams). In at least one example embodiment, the fifth consumable includes Vitamin A in an amount less than or equal to about 0.858 milligrams (e.g., less than or equal to about 0.85 milligrams, less than or equal to about 0.84 milligrams, less than or equal to about 0.83 milligrams, less than or equal to about 0.82 milligrams, less than or equal to about 0.81 milligrams, less than or equal to about 0.8 milligrams, less than or equal to about 0.79 milligrams, less than or equal to about 0.78 milligrams, less than or equal to about 0.77 milligrams, less than or equal to about 0.76 milligrams, less than or equal to about 0.75 milligrams, less than or equal to about 0.74 milligrams, less than or equal to about 0.73 milligrams, less than or equal to about 0.72 milligrams, or less than or equal to about 0.71 milligrams). In at least one example embodiment, the fifth consumable includes greater than or equal to about 0.702 milligrams to less than or equal to about 0.858 milligrams of Vitamin A. In at least one example embodiment, the fifth consumable includes about 0.78 milligrams of Vitamin A.

Vitamin C. In at least one example embodiment, the fifth consumable includes Vitamin C in an amount greater than or equal to about 54 milligrams (e.g., greater than or equal to about 55 milligrams, greater than or equal to about 56 milligrams, greater than or equal to about 57 milligrams, greater than or equal to about 58 milligrams, greater than or equal to about 59 milligrams, greater than or equal to about 60 milligrams, greater than or equal to about 61 milligrams, greater than or equal to about 62 milligrams, greater than or equal to about 63 milligrams, greater than or equal to about 64 milligrams, or greater than or equal to about 65 milligrams). In at least one example embodiment, the fifth consumable includes Vitamin C in an amount less than or equal to about 66 milligrams (e.g., less than or equal to about 65 milligrams, less than or equal to about 64 milligrams, less than or equal to about 63 milligrams, less than or equal to about 62 milligrams, less than or equal to about 61 milligrams, less than or equal to about 60 milligrams, less than or equal to about 59 milligrams, less than or equal to about 58 milligrams, less than or equal to about 57 milligrams, less than or equal to about 56 milligrams, or less than or equal to about 55 milligrams). In at least one example embodiment, the fifth consumable includes greater than or equal to about 54 milligrams to less than or equal to about 66 milligrams of Vitamin C. In at least one example embodiment, the fifth consumable includes about 60 milligrams of Vitamin C.

Vitamin B6. In at least one example embodiment, the fifth consumable includes Vitamin B6 in an amount greater than or equal to about 1.8 milligrams (e.g., greater than or equal to about 1.9 milligrams, greater than or equal to about 2 milligrams, or greater than or equal to about 2.1 milligrams). In at least one example embodiment, the fifth consumable includes Vitamin B6 in an amount less than or equal to about 2.2 milligrams (e.g., less than or equal to about 2.1 milligrams, less than or equal to about 2 milligrams, or less than or equal to about 1.9 milligrams). In at least one example embodiment, the fifth consumable includes greater than or equal to about 1.8 milligrams to less than or equal to about 2.2 milligrams of Vitamin B6. In at least one example embodiment, the fifth consumable includes about 2 milligrams of Vitamin B6.

Vitamin B12. In at least one example embodiment, the fifth consumable includes Vitamin B12 in an amount greater than or equal to about 0.0072 milligrams (e.g., greater than or equal to about 0.0073 milligrams, greater than or equal to about 0.0074 milligrams, greater than or equal to about 0.0075 milligrams, greater than or equal to about 0.0076 milligrams, greater than or equal to about 0.0077 milligrams, greater than or equal to about 0.0078 milligrams, greater than or equal to about 0.0079 milligrams, greater than or equal to about 0.008 milligrams, greater than or equal to about 0.0081 milligrams, greater than or equal to about 0.0082 milligrams, greater than or equal to about 0.0083 milligrams, greater than or equal to about 0.0084 milligrams, greater than or equal to about 0.0085 milligrams, greater than or equal to about 0.0086 milligrams, or greater than or equal to about 0.0087 milligrams). In at least one example embodiment, the fifth consumable includes Vitamin B12 in an amount less than or equal to about 0.0088 milligrams (e.g., less than or equal to about 0.0087 milligrams, less than or equal to about 0.0086 milligrams, less than or equal to about 0.0085 milligrams, less than or equal to about 0.0084 milligrams, less than or equal to about 0.0083 milligrams, less than or equal to about 0.0082 milligrams, less than or equal to about 0.0081 milligrams, less than or equal to about 0.008 milligrams, less than or equal to about 0.0079 milligrams, less than or equal to about 0.0078 milligrams, less than or equal to about 0.0077 milligrams, less than or equal to about 0.0076 milligrams, less than or equal to about 0.0075 milligrams, less than or equal to about 0.0074 milligrams, or less than or equal to about 0.0073 milligrams). In at least one example embodiment, the fifth consumable includes greater than or equal to about 0.0072 milligrams to less than or equal to about 0.0088 milligrams of Vitamin B12. In at least one example embodiment, the fifth consumable includes about 0.008 milligrams of Vitamin B12.

Choline Bitartrate. In at least one example embodiment, the fifth consumable includes choline bitartrate in an amount greater than or equal to about 405 milligrams (e.g., greater than or equal to about 410 milligrams, greater than or equal to about 420 milligrams, greater than or equal to about 430 milligrams, greater than or equal to about 440 milligrams, greater than or equal to about 450 milligrams, greater than or equal to about 460 milligrams, greater than or equal to about 470 milligrams, greater than or equal to about 480 milligrams, or greater than or equal to about 490 milligrams). In at least one example embodiment, the fifth consumable includes choline bitartrate in an amount less than or equal to about 495 milligrams (e.g., less than or equal to about 490 milligrams, less than or equal to about 480 milligrams, less than or equal to about 470 milligrams, less than or equal to about 460 milligrams, less than or equal to about 450 milligrams, less than or equal to about 440 milligrams, less than or equal to about 430 milligrams, less than or equal to about 420 milligrams, or less than or equal to about 410 milligrams). In at least one example embodiment, the fifth consumable includes greater than or equal to about 405 milligrams to less than or equal to about 495 milligrams of choline bitartrate. In at least one example embodiment, the fifth consumable includes about 450 milligrams of choline bitartrate.

Zinc. In at least one example embodiment, the fifth consumable includes zinc in an amount greater than or equal to about 9.9 milligrams (e.g., greater than or equal to about 10 milligrams, greater than or equal to about 10.5 milligrams, greater than or equal to about 11 milligrams, greater than or equal to about 11.5 milligrams, or greater than or equal to about 12 milligrams). In at least one example embodiment, the fifth consumable includes zinc in an amount less than or equal to about 12.1 milligrams (e.g., less than or equal to about 12 milligrams, less than or equal to about 11.5 milligrams, less than or equal to about 11 milligrams, less than or equal to about 10.5 milligrams, or less than or equal to about 10 milligrams). In at least one example embodiment, the fifth consumable includes greater than or equal to about 9.9 milligrams to less than or equal to about 12.1 milligrams of zinc. In at least one example embodiment, the fifth consumable includes about 11 milligrams of zinc.

Folic Acid. In at least one example embodiment, the fifth consumable includes folic acid in an amount greater than or equal to about 0.72 milligrams (e.g., greater than or equal to about 0.73 milligrams, greater than or equal to about 0.74 milligrams, greater than or equal to about 0.75 milligrams, greater than or equal to about 0.76 milligrams, greater than or equal to about 0.77 milligrams, greater than or equal to about 0.78 milligrams, greater than or equal to about 0.79 milligrams, greater than or equal to about 0.8 milligrams, greater than or equal to about 0.81 milligrams, greater than or equal to about 0.82 milligrams, greater than or equal to about 0.83 milligrams, greater than or equal to about 0.84 milligrams, greater than or equal to about 0.85 milligrams, greater than or equal to about 0.86 milligrams, or greater than or equal to about 0.87 milligrams). In at least one example embodiment, the fifth consumable includes folic acid in an amount less than or equal to about 0.88 milligrams (e.g., less than or equal to about 0.87 milligrams, less than or equal to about 0.86 milligrams, less than or equal to about 0.85 milligrams, less than or equal to about 0.84 milligrams, less than or equal to about 0.83 milligrams, less than or equal to about 0.82 milligrams, less than or equal to about 0.81 milligrams, less than or equal to about 0.8 milligrams, less than or equal to about 0.79 milligrams, less than or equal to about 0.78 milligrams, less than or equal to about 0.77 milligrams, less than or equal to about 0.76 milligrams, less than or equal to about 0.75 milligrams, less than or equal to about 0.74 milligrams, or less than or equal to about 0.73 milligrams). In at least one example embodiment, the fifth consumable includes greater than or equal to about 0.72 milligrams to less than or equal to about 0.88 milligrams of folic acid. In at least one example embodiment, the fifth consumable includes about 0.8 milligrams of folic acid.

Copper. In at least one example embodiment, the fifth consumable includes copper in an amount greater than or equal to about 0.9 milligrams (e.g., greater than or equal to about 0.95 milligrams, greater than or equal to about 1 milligrams, or greater than or equal to about 1.05 milligrams). In at least one example embodiment, the fifth consumable includes copper in an amount less than or equal to about 1.1 milligrams (e.g., less than or equal to about 1.05 milligrams, less than or equal to about 1 milligrams, or less than or equal to about 0.95 milligrams). In at least one example embodiment, the fifth consumable includes greater than or equal to about 0.9 milligrams to less than or equal to about 1.1 milligrams of copper. In at least one example embodiment, the fifth consumable includes about 1 milligrams of copper.

The foregoing description of the embodiments has been provided for purposes of illustration and description. It is not intended to be exhaustive or to limit the disclosure. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the disclosure, and all such modifications are intended to be included within the scope of the disclosure.

What is claimed is:

1. A dietary supplement for human consumption, the dietary supplement comprising:
a xanthophyll carotenoid including lutein, zeaxanthin, and meso-zeaxanthin, the dietary supplement including:
greater than or equal to about 0.27 weight percent to less than or equal to about 0.47 weight percent of lutein,
greater than or equal to about 0.066 weight percent to less than or equal to about 0.84 weight percent of zeaxanthin, and
greater than or equal to about 0.27 weight percent to less than or equal to about 0.47 weight percent of meso-zeaxanthin.

2. The dietary supplement of claim 1, wherein the weight ratio of lutein:zeaxanthin:meso-zeaxanthin is about 5:1:5.

3. The dietary supplement of claim 1, wherein the dietary supplement includes greater than or equal to about 19.8 milligrams to less than or equal to about 24.2 milligrams of the xanthophyll carotenoid.

4. The dietary supplement of claim 1, wherein the dietary supplement includes:
greater than or equal to about 2 milligrams to less than or equal to about 11 milligrams of lutein;
greater than or equal to about 0.4 milligrams to less than or equal to about 2 milligrams of zeaxanthin; and
greater than or equal to about 2 milligrams to less than or equal to about 10 milligrams of meso-zeaxanthin.

5. The dietary supplement of claim 1, wherein the dietary supplement further includes Vitamin A, Vitamin C, Vitamin D3, Vitamin E, thiamin, riboflavin, niacin, Vitamin B6, folic acid, Vitamin B12, biotin, panthothenic acid, calcium, iron, iodine, magnesium, zinc, copper, choline, omega-3, selenium, manganese, ginger, or any combination thereof.

6. The dietary supplement of claim 5, wherein the dietary supplement includes:
greater than or equal to about 0.702 milligrams to less than or equal to about 0.858 milligrams of Vitamin A;
greater than or equal to about 54 milligrams to less than or equal to about 66 milligrams of Vitamin C;
greater than or equal to about 0.03375 milligrams to less than or equal to about 0.04125 milligrams of Vitamin D3;
greater than or equal to about 18 milligrams to less than or equal to about 22 milligrams of Vitamin E;
greater than or equal to about 1.53 milligrams to less than or equal to about 1.87 milligrams of thiamin;
greater than or equal to about 1.98 milligrams to less than or equal to about 2.02 milligrams of riboflavin;
greater than or equal to about 18 milligrams to less than or equal to about 22 milligrams of niacin;
greater than or equal to about 1.8 milligrams to less than or equal to about 2.2 milligrams of Vitamin B6;
greater than or equal to about 0.72 milligrams to less than or equal to about 0.88 milligrams of folic acid;
greater than or equal to about 0.0072 milligrams to less than or equal to about 0.0088 milligrams of Vitamin B12;
greater than or equal to about 0.045 milligrams to less than or equal to about 0.055 milligrams of biotin;
greater than or equal to about 9 milligrams to less than or equal to about 11 milligrams of panthothenic acid;
greater than or equal to about 450 milligrams to less than or equal to about 550 milligrams of calcium;
greater than or equal to about 16.2 milligrams to less than or equal to about 19.8 milligrams of iron;
greater than or equal to about 135 milligrams to less than or equal to about 165 milligrams of iodine;
greater than or equal to about 45 milligrams to less than or equal to about 55 milligrams of magnesium;
greater than or equal to about 0.9 milligrams to less than or equal to about 1.1 milligrams of zinc;
greater than or equal to about 0.9 milligrams to less than or equal to about 1.1 milligrams of copper;
greater than or equal to about 405 milligrams to less than or equal to about 495 milligrams of choline;
greater than or equal to about 900 milligrams to less than or equal to about 1,100 milligrams of omega-3;
greater than or equal to about 0.054 milligrams to less than or equal to about 0.066 milligrams of selenium;
greater than or equal to about 1.98 milligrams to less than or equal to about 2.02 milligrams of manganese; and
greater than or equal to about 339 milligrams to less than or equal to about 411 milligrams of ginger.

7. The dietary supplement of claim 5, wherein the omega-3 includes docosahexaenoic acid, eicosapentaenoic acid, or a combination of docosahexaenoic acid and eicosapentaenoic acid.

8. The dietary supplement of claim 7, wherein the dietary supplement includes:
about 0.78 milligrams of Vitamin A;
about 60 milligrams of Vitamin C;
about 0.0375 milligrams of Vitamin D3;
about 20 milligrams of Vitamin E,
about 1.7 milligrams of thiamin;
about 2 milligrams of riboflavin;
about 20 milligrams of niacin;
about 2 milligrams of Vitamin B6;
about 0.80 milligrams of folic acid;
about 0.008 milligrams of Vitamin B12;
about 0.05 milligrams of biotin;
about 10 milligrams of panthothenic acid;
about 500 milligrams of calcium;
about 18 milligrams of iron;
about 150 milligrams of iodine;
about 50 milligrams of magnesium,
about 11 milligrams of zinc;
about 1 milligram of copper;
about 450 milligrams of choline;
about 500 milligrams of docosahexaenoic acid;
about 500 milligrams of eicosapentaenoic acid;
about 0.06 milligrams of selenium;
about 2 milligrams of manganese; and
about 375 milligrams of ginger.

9. The dietary supplement of claim 1, wherein the dietary supplement further includes greater than or equal to about 100 milligrams to less than or equal to about 300 milligrams of a filler.

10. The dietary supplement of claim 9, wherein the filler includes sunflower oil, micro-crystalline cellulose, silica, or any combination thereof.

11. The dietary supplement of claim 1, wherein the dietary supplement includes a first consumable, the first consumable including lutein, meso-zeaxanthin, zeaxanthin, or any combination thereof.

12. The dietary supplement of claim 11, wherein the first consumable includes:
greater than or equal to about 2 milligrams to less than or equal to about 11 milligrams of lutein;
greater than or equal to about 2 milligrams to less than or equal to about 10 milligrams of meso-zeaxanthin; and
greater than or equal to about 0.4 milligrams to less than or equal to about 2 milligrams of zeaxanthin.

13. The dietary supplement of claim 11, wherein the first consumable further includes omega-3, Vitamin E, or a combination of omega-3 and Vitamin E.

14. The dietary supplement of claim 13, wherein the first consumable includes:
greater than or equal to about 450 milligrams to less than or equal to about 550 milligrams of omega-3; and
greater than or equal to about 9 milligrams to less than or equal to about 11 milligrams of Vitamin E.

15. The dietary supplement of claim 11, wherein the dietary supplement includes a second consumable, the second consumable including omega-3, Vitamin E, or a combination of omega-3 and Vitamin E.

16. The dietary supplement of claim 15, wherein the second consumable includes:
greater than or equal to about 450 milligrams to less than or equal to about 550 milligrams of omega-3; and
greater than or equal to about 9 milligrams to less than or equal to about 11 milligrams of Vitamin E.

17. The dietary supplement of claim 15, wherein the dietary supplement includes a third consumable, the third consumable including Vitamin D3, manganese, calcium, or any combination thereof.

18. The dietary supplement of claim 17, wherein the third consumable includes:
greater than or equal to about 0.03375 milligrams to less than or equal to about 0.04125 milligrams of Vitamin D3;
greater than or equal to about 1.8 milligrams to less than or equal to about 2.2 milligrams of manganese; and
greater than or equal to about 450 milligrams to less than or equal to about 550 milligrams of calcium.

19. The dietary supplement of claim 17, wherein the dietary supplement includes a fourth consumable, the fourth consumable including Vitamin B1, Vitamin B2, niacin, biotin, pantothenic acid, iodine, magnesium, selenium, ginger, or any combination thereof.

20. The dietary supplement of claim 19, wherein the fourth consumable includes:
greater than or equal to about 1.53 milligrams to less than or equal to about 1.87 milligrams of Vitamin B1;
greater than or equal to about 1.8 milligrams to less than or equal to about 2.2 milligrams of Vitamin B2;
greater than or equal to about 18 milligrams to less than or equal to about 22 milligrams of niacin;
greater than or equal to about 0.045 milligrams to less than or equal to about 0.055 milligrams of biotin;
greater than or equal to about 9 milligrams to less than or equal to about 11 milligrams of pantothenic acid;
greater than or equal to about 135 milligrams to less than or equal to about 165 milligrams of iodine;
greater than or equal to about 45 milligrams to less than or equal to about 55 milligrams of magnesium;
greater than or equal to about 0.054 milligrams to less than or equal to about 0.066 milligrams of selenium; and
greater than or equal to about 315 milligrams to less than or equal to about 385 milligrams of ginger.

21. The dietary supplement of claim 19, wherein the dietary supplement includes a fifth consumable, the fifth consumable including iron, Vitamin A, Vitamin C, Vitamin B6, Vitamin B12, choline, zinc, folic acid, copper, or any combination thereof.

22. The dietary supplement of claim 21, wherein the dietary supplement includes:
greater than or equal to about 16.2 milligrams to less than or equal to about 19.8 milligrams of iron;
greater than or equal to about 0.702 milligrams to less than or equal to about 0.858 milligrams of Vitamin A;
greater than or equal to about 54 milligrams to less than or equal to about 66 milligrams of Vitamin C;
greater than or equal to about 1.8 milligrams to less than or equal to about 2.2 milligrams of Vitamin B6;
greater than or equal to about 0.0072 milligrams to less than or equal to about 0.0088 milligrams of Vitamin B12;
greater than or equal to about 405 milligrams to less than or equal to about 495 milligrams of choline;
greater than or equal to about 9.9 milligrams to less than or equal to about 12.1 milligrams of zinc;
greater than or equal to about 0.72 milligrams to less than or equal to about 0.88 milligrams of folic acid; and
greater than or equal to about 0.9 milligrams to less than or equal to about 1.1 milligrams of copper.
